(19) United States
(12) Patent Application Publication
(10) Pub. No.: US 2006/0202197 A1
(43) Pub. Date: Sep. 14, 2006

(54) Platinum complex and light-emitting device

(75) Inventors: Yuji Nakayama, Kanagawa (JP); Hisanori Ito, Kanagawa (JP); Takeshi Iwata, Kanagawa (JP); Junji Nakamura, Kanagawa (JP); Yoshimasa Matsushima, Kanagawa (JP)

Correspondence Address: MCDERMOTT WILL & EMERY LLP, 600 13TH STREET, N.W., WASHINGTON, DC, 20005-3096, US

(73) Assignee: TAKASAGO INTERNATIONAL CORPORATION

(21) Appl. No.: 11/363,182

(22) Filed: Feb. 28, 2006

(30) Foreign Application Priority Data
Feb. 28, 2005 (JP) 2005-053502

(51) Int. Cl. C07F 15/00; H01L 29/08; H01J 1/62

(52) U.S. Cl. 257/40; 546/2; 548/101; 544/179; 544/181; 544/225; 313/504; 556/137

(57) ABSTRACT

A platinum complex represented by the general formula 1, useful as a phosphorescence emission material, a tetradentate ligand useful for synthesizing the platinum complex, and a light-emitting device containing at least one of the platinum complex.

In the general formula 1, two of the rings A, B, C, and D each independently represent an aromatic ring or an aromatic heterocyclic ring, while the other two rings each represent a nitrogen-containing heterocyclic ring; R^{A-D} represent the substituents; each the rings A and B, the rings B and C, and the rings C and D may be bound to each other to form a fused ring independently via the substituent R^{A-D}; X^{A-D} each represent a carbon atom or nitrogen atom; Q represents a bivalent atom or atomic group; Y represents a carbon or nitrogen atom; and n is an integer of 0 to 3.

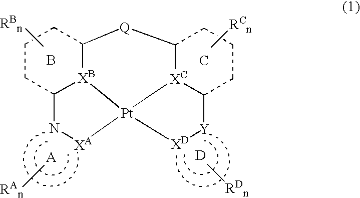

### BACKGROUND OF THE INVENTION

1. Field of the Invention

The present invention relates to a new platinum complex useful, for example, as a light-emitting material and a light-emitting device using the complex. Further, the present invention, minutely, relates to a new platinum complex usable, for example, as a light-emitting material in the fields such as a display device, a display, a backlight, an electrophotographic machine, an illumination light source, a recording light source, an exposure light source, a reading light source, a sign and mark, a signboard, and interior goods; and a light-emitting device using the complex.

2. Description of the Related Art

Researches and developments on various display devices are intensively carried out recently, and among them, an organic electroluminescent device (hereinafter, referred to as “organic EL device”), which emits high-brightness light at low voltage, is attracting attention as a promising next-generation display device. The organic EL device is faster in response speed than liquid crystal devices so far used and emits a selfluminous light, and thus, does not demand backlight like the conventional liquid crystal devices and allows production of an extremely thinner flat panel display. The organic EL device is a light-emitting device utilizing an electroluminescent phenomenon. That is the same in principle as conventional LED's, but because it uses an organic compound as its light-emitting material, it is characteristic in that the degree of freedom on the film production is greater. For that reason, expected are applications not only as flat panel displays but also as flexible display devices such as an electronic paper and an electronic poster.

An example of the organic EL device using an organic compound as the light-emitting material so far reported is a device having a multilayer thin film prepared by a vapor deposition. According to the report, light-emitting characteristics of such the organic EL device are improved significantly compared to those of conventional single-layer devices, by using tris(8-hydroxyquinolinato-O,N)-aluminum (Alq₃) as its electron transporting material and laminating it with a hole transporting material (e.g., aromatic amine compound). Although studies for application of such an organic EL device to multi-color display are eagerly carried out recently, it is still necessary to improve the light-emitting characteristics in the three primary colors of light, red, green and blue and the emission efficiency thereof for successful development of a high-performance multi-color display.

Use of a phosphorescent material in the light-emitting layer of the organic EL device was proposed as the means for improving the light-emitting characteristics. Phosphorescence emission is a phenomenon of light emission in the relaxation process from triplet excited state, but, because the relaxation process is normally conducted by thermal deactivation, it is not possible generally to observe the phosphorescence emission at room temperature. The theoretical maximum internal quantum efficiency of light-emitting materials using an emission phenomenon in the relaxation process from singlet excited state, i.e., fluorescence emission, does not exceed 25% in organic EL devices, because the ratio of the singlet to the triplet in the excited state of the light-emitting material is always 25 to 75. On the other hand, if a substance able of observing phosphorescence emission at room temperature is used as a light-emitting material, it is possible to raise the theoretical maximum internal quantum efficiency to 100% by taking into consideration the intersystem crossing from singlet to triplet excited state and to increase the efficiency of the organic EL device to a significantly greater degree.

As described above, it is difficult to get phosphorescence emission from an organic compound at room temperature or higher, because of prohibited intersystem crossing and concurrent thermal deactivation in the triplet relaxation process. However the phosphorescence emission is allowed occasionally in an organic compound containing a heavy metal, i.e., metal complex, because of the spin-orbit interaction resulting from the heavy-atom effect. As the organic EL devices containing a metal complex having such properties as the phosphorescent material, devices using various complexes having iridium as the heavy metal have been so far developed. In addition, there are some scattered reports on devices containing complexes having platinum as the heavy metal recently.

An organic EL device containing a platinum complex as the red phosphorescent material reported in the early stage was an device using (2,3,7,8,12,13,17,18-octaethyl-21H,23H-porphinato-N,N,N,N)-platinum (II) in its light-emitting layer (Thompson Mark E. et al., U.S. Pat. No. 6,303,238 B2 (Patent Document 1)). The platinum complex was a red phosphorescence-emitting substance showing high color purity, but the maximum external quantum efficiency thereof was approximately 4%, lower than the theoretical limit of 5% in external quantum efficiency of fluorescence-emitting materials, and thus, there is a need for further improvement in its luminous efficiency. However, it is extremely difficult to synthesize the derivatives used for improvement in luminous efficiency, because the ligand is a macrocyclic compound.

On the other hand, there was reported that an ortho-metalated platinum complex, in which a compound having an arylpyridine skeleton was used as the ligand and platinum as the heavy atom, was useful as a phosphorescence-emitting material (Igarashi Tatsuya., JP 2001-181917 A (Patent Document 2)). In addition, there was also reported that a platinum complex in which a bipyridine/biaryl skeleton compound was used as the ligand (Tsuboyama Akira et al., US 2002/0068190 A1 (Patent Document 3)). The compounds described in Patent Documents 2 and 3 are more advantageous compared with the compound described in Patent Document 1 in the diversity on the synthesis of derivatives as these compounds are platinum complexes having a monodentate or bidentate ligand. However, as the chelating effect that participates in the interaction and bonding force between metal and ligand increases drastically with increase in the number of the conformation in a single ligand, these compounds described in Patent Documents 2 and 3 are far lower in the physical and chemical stability of complex than the platinum complexes described in Patent Document 1 from a viewpoint of chelating effect. In addition, platinum complexes having a monodentate or bidentate ligand have a particular problem that cis- and trans-coordinated isomers are formed. Therefore it is difficult to control the structure, that is, to adjust the ratio of cis- and trans-coordinated isomers of these platinum complexes.

A platinum complex using a tetradentate ligand obtained by introducing a phenol group into a bipyridine or phenanthroline skeleton was reported recently from a viewpoint of overcoming such a problem (Yong-Yue Lin et al., Chemistry European Journal, 6(2003), 1264-1272 (Non-patent Document 1)). The compound described in Non-patent Document 1 is a platinum complex very superior in thermal stability (decomposition point: >400° C.). As the ligand is a noncyclic compound, it is relatively easy to synthesize the derivatives thereof, although a phenol group is contained in the derivatives. However, the maximum power efficiency thereof when applied to an organic EL device was still 1 lm/W or less, and there is a need for significant improvement in luminous efficiency for application in a next-generation display device.

As described above, various studies are in progress for commercialization of next-generation display devices now, and among them, organic EL devices using a phosphorescence-emitting material are particularly attracting greater attention for improvement in the properties of the devices. However, the research is fairly under way, and there are still many problems to be solved such as improvement in the light-emitting characteristics, luminous efficiency, and color purity of device as well as optimization of the structure. To solve these problems, there exists a need for development of a new phosphorescence-emitting material and further an efficient supplying method of the materials.

### SUMMARY OF THE INVENTION

An object of the present invention, which was made in view of the problems above, is to provide a platinum complex extremely favorable in thermal stability, light-emitting characteristics and luminous efficiency and useful, for example, as a material for light-emitting devices, and a light-emitting device using the complex, that is superior in light-emitting characteristics and luminous efficiency.

After intensive studies to overcome the problems above, the present inventors have found that a platinum complex represented by the following General Formula (1) (hereinafter, referred to as “platinum complex of the present invention”) was superior in thermal stability, light-emitting characteristics and luminous efficiency. After further studies for preparation of devices based on the finding, they also found that the platinum complex was quite favorable as a phosphorescence-emitting material for light-emitting devices, and completed the present invention.

Accordingly, the present invention relates to a platinum complex represented by General Formula (1): 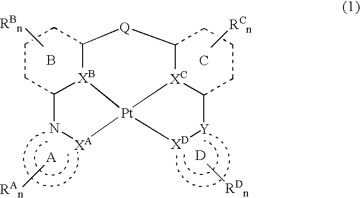
wherein two of the rings A, B, C, and D each independently represent an aromatic ring or an aromatic heterocyclic ring, while the other two rings each independently represent a nitrogen-containing heterocyclic ring; each of the rings B and C is always a six-membered ring independently of the kind of its ring; R^{A}, R^{B}, R^{C}, and R^{D} respectively represent substituents on the rings A, B, C, and D; the rings A and B, the rings B and C, and the rings C and D each may be bound each other via the substituent R^{A}, R^{B}, R^{C} or R^{D} to form a fused ring independently; X^{A}, X^{B}, X^{C}, and X^{D} each independently represent a carbon atom that can be bound with the platinum atom by a covalent bond or a nitrogen atom that can be bound with the platinum atom by a covalent bond when the corresponding ring is an aromatic ring or an aromatic heterocyclic ring, and a nitrogen atom that can be bound with the platinum atom by a coordinate bond when the corresponding ring is a nitrogen-containing heterocyclic ring; Q represents a bivalent atom or atomic group bridging the rings B and C; the ring B and Q, and the ring C and Q each independently may be bound each other via a substituent R^{B} or R^{C} to form a fused ring; Y represents a carbon atom or a nitrogen atom; n is an integer of 0 to 3; and when n is 2 or more, the groups R^{A}, the groups R^{B}, the groups R^{C}, and the groups R^{D} each independently may be bound each other to form a fused ring.

In addition, the present invention relates to a light-emitting device containing one or more of the platinum complexes represented by General Formula (1) above.

In addition, the present invention relates to a compound represented by General Formula (2): 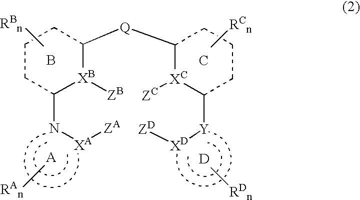
wherein two of the rings A, B, C, and D each independently represent an aromatic ring or an aromatic heterocyclic ring, while the other two rings each independently represent a nitrogen-containing heterocyclic ring; each of the rings B and C is always a six-membered ring independently of the kind of its ring; R^{A}, R^{B}, R^{C}, and R^{D} respectively represent substituents on the rings A, B, C, and D; the rings A and B, the rings B and C, and the rings C and D each may be bound each other via the substituent R^{A}, R^{B}, R^{C}, or R^{D} to form a fused ring independently; X^{A}, X^{B}, X^{C}, and X^{D} each independently represent a carbon atom or a nitrogen atom when the corresponding ring is an aromatic ring or an aromatic heterocyclic ring, and a nitrogen atom when the corresponding ring is a nitrogen-containing heterocyclic ring; Z^{A}, Z^{B}, Z^{C}, and Z^{D} each represent a hydrogen atom when the corresponding X is a carbon atom, and a hydrogen atom or a lone electron pair when the corresponding X is a nitrogen atom; Q represents a bivalent atom or atomic group bridging the rings B and C; and the ring B and Q, and the ring C and Q each independently may be bound each other via a substituent R^{B} or R^{C} to form a fused ring; Y represents a carbon atom or a nitrogen atom; n is an integer of 0 to 3; and when n is 2 or more, the groups R^{A}, the groups R^{B}, the groups R^{C}, and the groups R^{D} each may be independently bound each other to form a fused ring.

The platinum complex represented by General Formula (1) of the present invention is superior in thermal stability, light-emitting characteristics and luminous efficiency, and useful as a phosphorescence-emitting material being able to be used favorably in various light-emitting devices including organic EL devices. In addition, the light-emitting device containing the platinum complex of the present invention is superior in light-emitting characteristics and luminous efficiency, and emits light having various emission colors in a wide wavelength range from shorter wavelength (blue) to longer wavelength (red) depending on the platinum complex used. Therefore it is useful as a light-emitting device that can be used favorably in various display devices. Further, the compound represented by General Formula (2) is useful as a tetradentate ligand for use in synthesis of metal complexes including the platinum complexes represented by General Formula (1).

Favorable results obtained in the present invention seem to be because of the following reasons. That is, for example, as the platinum complexes described in Patent Document 1 are compounds having a tetradentate ligand, they are superior in fastness, but are extremely difficult to synthesize the derivatives thereof because the ligand is a macrocyclic compound. In addition, the compounds are still poorer in luminous efficiency and those reported are only longer-wavelength (red) phosphorescence-emitting materials. In contrast, on the platinum complex represented by General Formula (1) of the present invention, various kinds of derivatives can be synthesized by the combination of the rings A, B, C, and D and the bridging unit Q, and further a phosphorescence-emitting material which emits high efficient light in a wide wavelength region from shorter wavelength (blue) to longer wavelength (red) can be prepared by joining these partial structures properly.

In addition, the compounds described in Patent Documents 2 and 3, platinum complexes having a monodentate or bidentate ligand, are advantageous in syntheses of derivatives for improvement in physical properties, but far inferior in the physical and chemical stabilities of complex compared to the platinum complexes described in Patent Document 1 from a viewpoint of the chelating effect. Further, it is quite difficult to control the structure of the cis- and trans-coordinated isomers inherent to the platinum complexes having a monodentate or bidentate ligand. In contrast, the platinum complexes of tetradentate coordination of the present invention show a thermal stability equivalent to or higher than that of the compounds described in Patent Document 1 (380 to 460° C.). The tetradentate coordination also prohibits the coordination isomerization phenomenon, and allows production of a complex with definite composition of the cis- and trans-coordinated isomers.

The compounds described in Non-patent Document 1, platinum complexes having a noncyclic tetradentate ligand, are also improved in the fastness, stability and easiness in synthesis of the derivatives compared with the compounds described in Patent Documents above, but have a power efficiency of 1 lm/W or less when applied to an organic EL device, and thus are not applicable, for example, to display devices. In contrast, the platinum complex of the present invention is improved by 5 to 10 times in the power efficiency compared with compounds described in Non-patent Document 1 when applied to an organic EL device, and on the luminous efficiency, the external quantum efficiency of the fluorescence-emitting material is much higher than its theoretical limit of 5%.

Characteristically, on the platinum complexes of the present invention, various kinds of derivatives can be synthesized easily as a single coordination isomer, and therefore they are applicable to various high-efficiency phosphorescence-emitting materials which emit the light in a wide wavelength range from shorter to longer wavelength; and each of them has high physical and chemical stabilities despite the diversity of the derivatives. Further, it will be apparent from physical properties of the platinum complexes determined in the Examples that they have superior luminous efficiency, wider light-emitting wavelengths, and higher stabilities as compared with conventional platinum complexes.

### BREIF DESCRIPTION OF THE DRAWING

FIG. 1 is a view illustrating the configuration of the organic EL device used in Examples.

### DESCRIPTION OF THE PREFERRED EMBODIMENTS

Hereinafter, the platinum complex represented by General Formula (1) and the compound represented by General Formula (2) of the present invention will be described in more detail.

As shown in General Formula (1) above, the platinum complex of the present invention is a platinum complex having a tetradentate ligand containing rings A, B, C, and D wherein the ring B and the ring C are bridged by Q.

In addition, the compound represented by General Formula (2) of the present invention is a compound wherein the ring B and the ring C are bridged by Q. The compound represented by General Formula (2) is a favorable compound as a tetradentate ligand used in the synthesis of metal complexes including a platinum complex.

Hereinafter, both the compounds represented by General Formulae (1) and (2) will be referred to simply as the “compounds of the present invention”.

In the compounds of the present invention, two of the rings A, B, C, and D each independently represent an aromatic ring or an aromatic heterocyclic ring that may have a substituent R^{A}, R^{B}, R^{C}, or R^{D}; and the other two represent a nitrogen-containing heterocyclic ring that may have a substituent R^{A}, R^{B}, R^{C}, or R^{D}. In General Formulae (1) and (2), each the rings B and C is always a six-membered ring, independently of the kind of the ring. Each of the rings A and B, rings B and C, and rings C and D may be bound each other independently via a substituent group R^{A}, R^{B}, R^{C} or R^{D} to form a fused ring. In addition, the ring B and the Q, and the ring C and the Q may be bound to each other independently via a substituent R^{B} or R^{C} to form a fused ring.

The aromatic ring or the aromatic heterocyclic ring constituting the rings A to D in the compounds of the present invention is not particularly limited as long as it is an aromatic ring or an aromatic heterocyclic ring. Preferred examples of the aromatic ring and aromatic heterocyclic ring of the rings A to D include benzene, furan, thiophene, selenophene, tellurophene, pyrrole, pyridine, pyridazine, pyrimidine, pyrazine, 1,2,3-triazine, 1,2,4-triazine, 1,2,3,4-tetrazine, oxazole, isoxazole, thiazole, isothiazole, pyrazole, imidazole, 1,2,3-oxadiazole, 1,2,5-oxadiazole, 1,2,3-thiadiazole, 1,2,5-thiadiazole, triazole and tetrazole rings shown below, and the like.

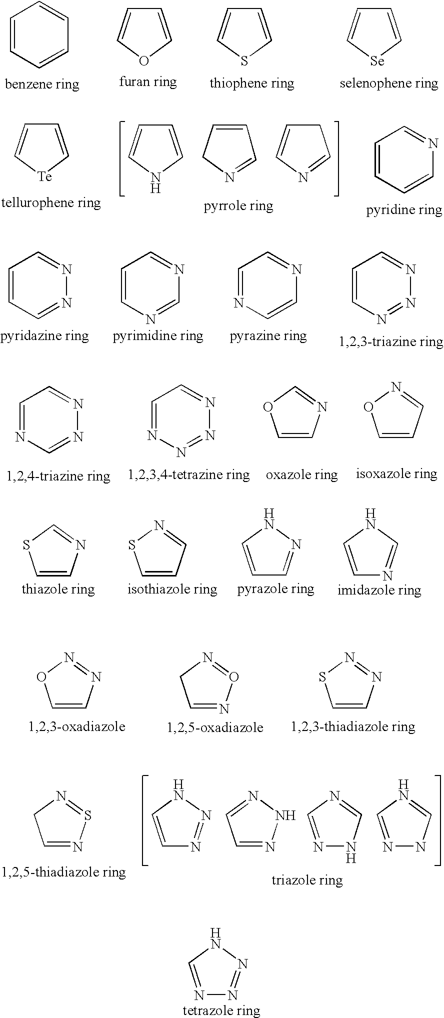

These rings may form a fused ring additionally with a ring selected from the group of rings described above. Examples of the fused rings include the benzologues of the respective rings; and typical examples thereof include naphthalene, anthracene, phenanthrene, chrysene, pyrene, benzofuran, isobenzofuran, thianaphthene, isothianaphthene, benzoselenophene, isobenzoselenophene, benzotellurophene, isobenzotellurophene, indole, isoindole, indolidine, quinoline, isoquinoline, cinnoline, phthalazine, quinazoline, quinoxaline, benzotriazine, benzotetrazine, benzoxazole, benzisoxazole, benzothiazole, benzisothiazole, indazole, benzimidazole, benzoxadiazole, benzothiadiazole ring and benzotriazole rings and the like.

More preferable examples of the aromatic ring and the aromatic heterocyclic ring include the benzene, naphthalene, furan, benzofuran, isobenzofuran, thiophene, thianaphthene, isothianaphthene, 1H-pyrrole, indole and isoindole rings shown by the structural formulae below, and the like.

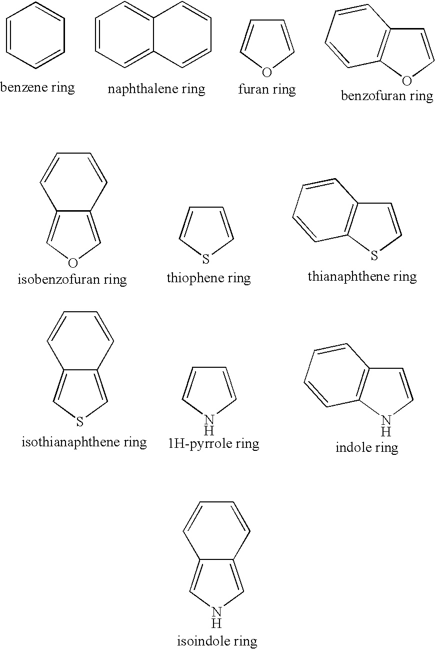

When the ring A or D is an aromatic ring or an aromatic heterocyclic ring in the compounds represented by General Formulae (1) and (2), typical examples of the preferable rings include 1H-pyrrole, indole, isoindole, pyrazole, 2H-indazole, imidazole, benzimidazole, triazole and tetrazole rings, and the like. Followings are examples when any one of these rings constitutes the ring A:

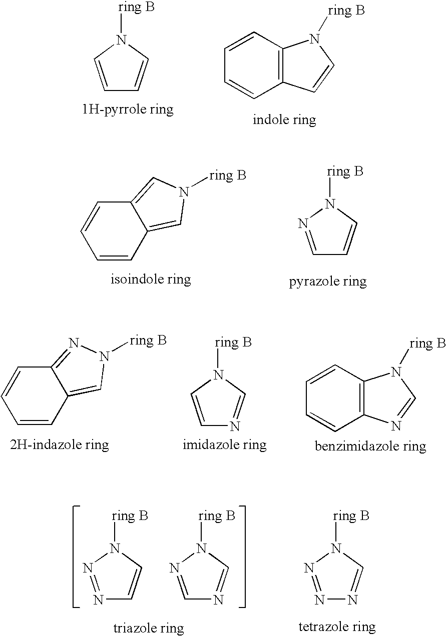

When each of the rings B and C is a six-membered aromatic or aromatic heterocyclic ring independently in the compounds represented by General Formulae (1) and (2), favorable examples of the rings include benzene, pyridine, pyridazine, pyrimidine and 1,2,3-triazine rings, and the like. A fused ring formed from a benzene ring and a suitable ring selected from the group of the aromatic rings and aromatic heterocyclic rings described above is also preferable, and typical examples of such rings include naphthalene, anthracene, phenanthrene, chrysene, pyrene, benzofuran, isobenzofuran, thianaphthene, isothianaphthene, benzoselenophene, isobenzoselenophene, benzotellurophene, isobenzotellurophene, indole, isoindole, indolidine, quinoline, isoquinoline, cinnoline, phthalazine, quinazoline, quinoxaline, benzotriazine, benzotetrazine, benzoxazole, benzisoxazole, benzothiazole, benzisothiazole, indazole, benzimidazole, benzoxadiazole, benzothiadiazole and benzotriazole rings, and the like. Examples of still more preferable rings include benzene, naphthalene, benzofuran, isobenzofuran, thianaphthene and isothianaphthene rings, and the like. In the present invention, as described above, six-membered rings include the fused rings of six-membered rings with another ring as well as six-membered rings. Examples when the ring exemplified above as the still more preferable rings constitutes the ring B are shown below:

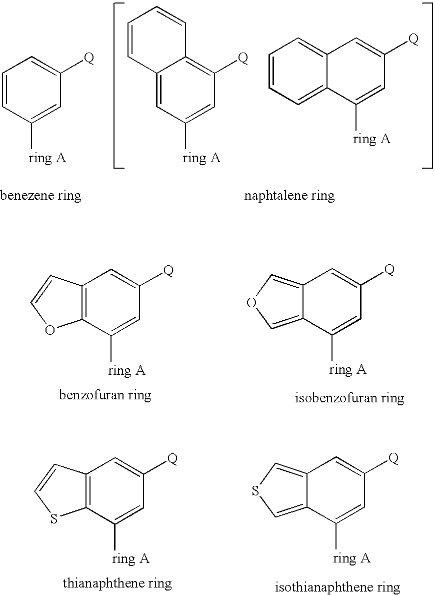

The nitrogen-containing heterocyclic ring constituting the rings A to D in the compounds of the present invention is not particularly limited, and preferable examples of the nitrogen-containing heterocyclic ring include the pyridine, pyridazine, pyrimidine, pyrazine, triazine, tetrazine, 2H-pyrrole, 3H-pyrrole, oxazole, isoxazole, thiazole, isothiazole, pyrazole, imidazole, oxadiazole, thiadiazole, triazole, oxatriazole, thiatriazole, tetrazole, 2H-3,4-dihydropyrrole, oxazoline, isooxazoline, thiazoline, isothiazoline, pyrazoline and imidazoline rings shown below, and the like.

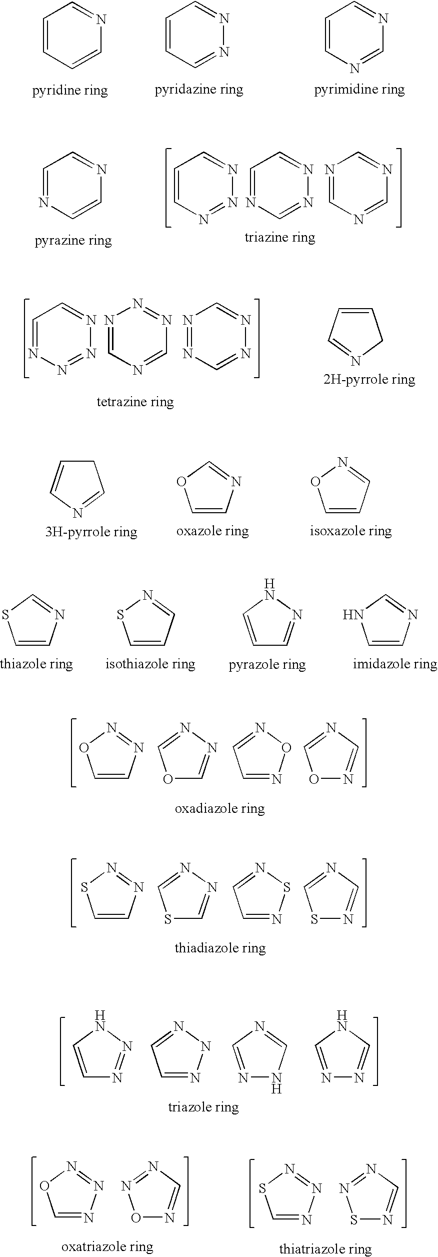

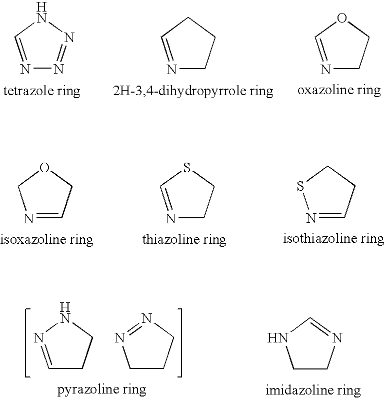

The rings above may be bound with a suitable ring selected from the group consisting of the aromatic rings and aromatic heterocyclic rings described above to form a fused ring. Examples of the fused ring include the benzologues of the respective rings, and typical examples thereof include quinoline, isoquinoline, cinnoline, phthalazine, quinazoline, quinoxaline, benzotriazine, benzotetrazine, 1H-isoindole, 3H-indole, benzoxazole, benzisoxazole, benzothiazole, benzisothiazole, indazole, benzimidazole, benzoxadiazole, benzothiadiazole, and benzotriazole rings, and the like.

More preferable examples of the nitrogen-containing heterocyclic ring include the pyridine, quinoline, isoquinoline, 2H-pyrrole, 1H-isoindole, 3H-pyrrole, 3H-indole, oxazole, benzoxazole, isoxazole, benzisoxazole, thiazole, benzothiazole, isothiazole, benzisothiazole, pyrazole, indazole, imidazole, benzimidazole, 2H-3,4-dihydropyrrole, oxazoline, isooxazoline, thiazoline, isothiazoline, pyrazoline and imidazoline rings shown by the structural formulae below, and the like.

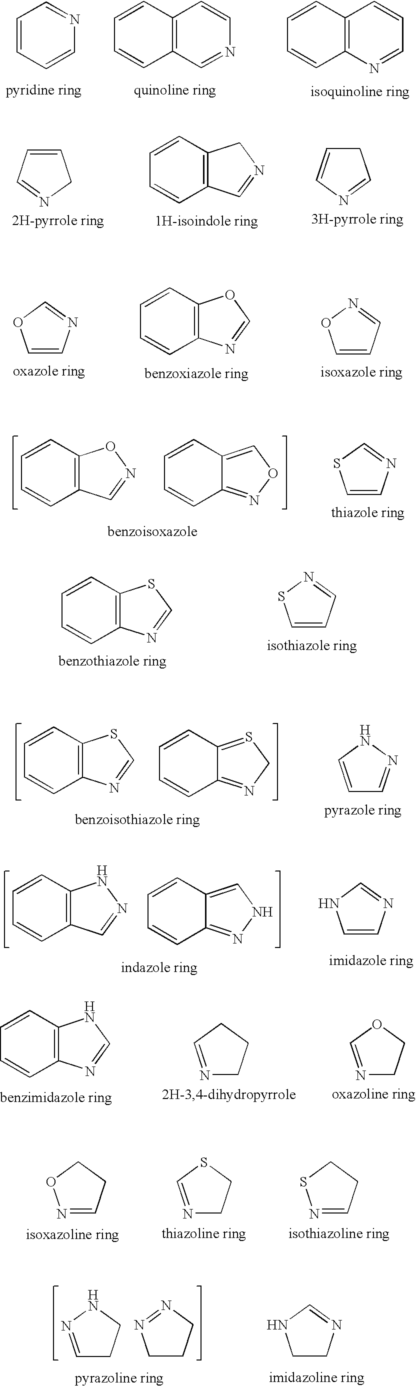

When the ring A in the compounds represented by General Formulae (1) and (2) is a nitrogen-containing heterocyclic ring, typical examples of the preferable rings thereof include the pyrazole, indazole, triazole, benzotriazole and tetrazole rings shown below, and the like; and more preferably examples thereof include pyrazole and indazole rings. When the ring D is a nitrogen-containing heterocyclic ring, typical examples of the preferable rings thereof include the preferable rings above, thiazole and pyridine rings, and the like.

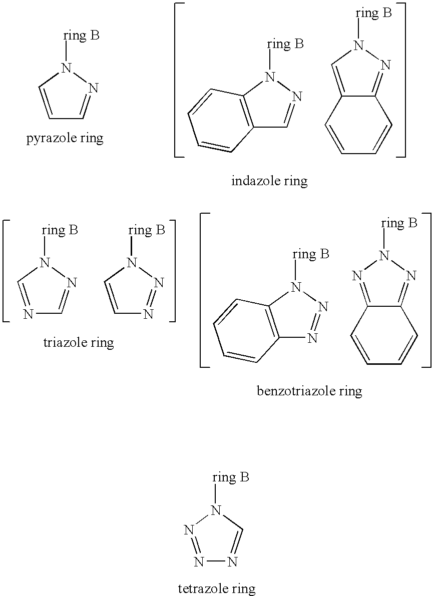

When the rings B and C in the compounds represented by General Formulae (1) and (2) each independently represent a nitrogen-containing heterocyclic ring, each of these rings is preferably a six-membered ring or the benzologue thereof, and examples thereof include the pyridine, isoquinoline, pyrimidine, quinazoline, pyrazine, 1,2,4-triazine, 1,3,5-triazine, and 1,2,3,5-tetrazine rings shown below, and the like; and more preferable rings thereof include pyridine and isoquinoline rings, and the like. Examples when the exemplified nitrogen-containing heterocyclic ring constitutes the ring B will be described below with reference to structural formulae.

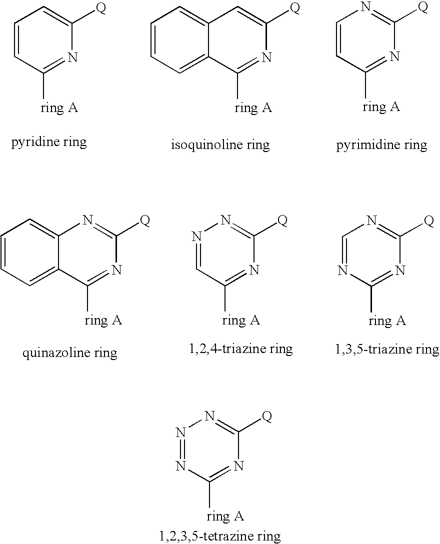

Each of the groups X (X^{A}, X^{B}, X^{C}, and X^{D}) in the compounds represented by General Formulae (1) and (2) represents a carbon atom that can be bound with the platinum atom covalently when the corresponding ring is an aromatic ring or an aromatic heterocyclic ring and a nitrogen atom that can be bound with the platinum atom by coordination when the corresponding ring is a nitrogen-containing heterocyclic ring.

The group Q in the compounds of the present invention represents a bivalent atom or atomic group bridging the rings B and C, and the bridging group Q will be described below in detail. Examples of the bivalent atom or atomic group include the oxy, thio, seleno, telluro, sulfinyl, sulfonyl, imino, phosphinidene, phosphinylidene, methylene, alkenylidene, carbonimidoyl, carbonyl, thiocarbonyl, silylene and borylene groups shown below. The states of the rings B and C being bridged by these preferable bivalent atoms or atomic groups are shown below. In the following Formulae, R represents a hydrogen atom or a substituent.

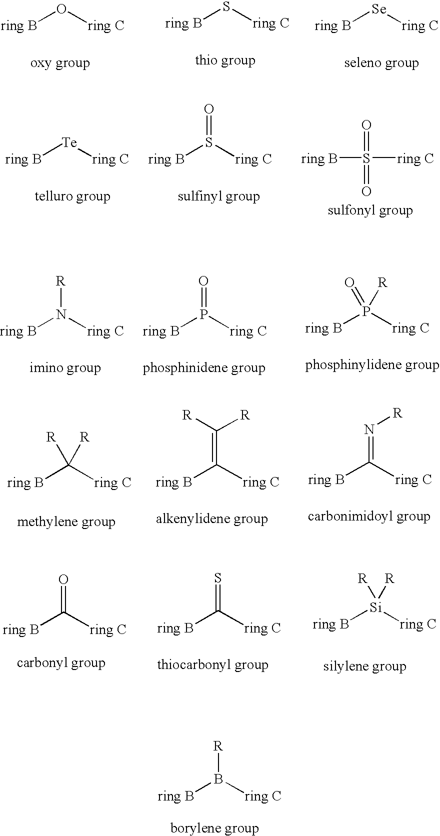

As shown in the Formulae above, the imino, phosphinidene, phosphinylidene, methylene, alkenylidene, carbonimidoyl, silylene and borylene groups may be substituted with a suitable substituent R described below. Examples of the substituted imino groups include imino groups in which the hydrogen atom on the nitrogen atom is substituted with a substituent such as imino-protecting group. The imino protecting group may be any one of the protecting groups described in known literatures (e.g., Protective Groups in Organic Synthesis, Third Ed., John Wiley & Sons, Inc. (Non-patent literature 2)), and typical examples thereof include alkyl, aryl, aralkyl, acyl, alkoxycarbonyl, aryloxycarbonyl, aralkyloxycarbonyl and sulfonyl groups, and the like. Typical examples of these groups are the same as those for substituents R^{A} to R^{D} described below, and detailed description thereof will not be repeated here.

Typical examples of the alkyl group-substituted imino groups include N-methylimino, N-ethylimino, N-isopropylimino and N-cyclohexylimino groups, and the like.

Typical examples of the aryl group-substituted imino group include N-phenylimino, N-(2,4,6-trimethylphenyl)imino, N-(2,6-diisopropylphenyl)imino, N-(3,5-di-tert-butylphenyl)imino, N-(1-naphthyl)imino, N-(2-naphthyl)imino and N-(9-anthryl)imino groups, and the like.

Typical examples of the aralkyl group-substituted imino groups include N-benzylimino and N-(1-phenylethyl)imino groups, and the like.

Typical examples of the acyl group-substituted imino groups include formylimino, acetylimino, propionylimino, acryloylimino, pivaloylimino, pentanoylimino, hexanoylimino and benzoylimino groups, and the like.

Typical examples of the alkoxycarbonyl group-substituted imino groups include methoxycarbonylimino, ethoxycarbonylimino, n-propoxycarbonylimino, n-butoxycarbonylimino, tert-butoxycarbonylimino, pentyloxycarbonylimino and hexyloxycarbonylimino groups, and the like.

Typical examples of the aryloxycarbonyl group-substituted imino groups include phenoxycarbonylimino and 2-naphthyloxycarbonylimino groups, and the like.

Typical examples of the aralkyloxycarbonyl group-substituted imino groups include a benzyloxycarbonylimino group and the like.

Typical examples of the sulfonyl group-substituted imino group include methanesulfonylimino and p-toluenesulfonylimino groups, and the like.

The phosphinidene group that may have a substituent group is, for example, a phosphinidene group in which the hydrogen atom on the phosphorus atom is substituted with a substituent such as a hydrocarbyl group; and typical examples thereof include methylphosphinidene, ethylphosphinidene, isopropylphosphinidene, phenylphosphinidene and benzylphosphinidene groups, and the like.

The phosphinylidene group that may have a substituent group is, for example, a phosphinylidene group in which the hydrogen atom on the phosphorus atom is substituted with a substituent such as a hydrocarbyl group; and typical examples thereof include methylphosphinylidene, ethylphosphinylidene, isopropylphosphinylidene, phenylphosphinylidene and benzylphosphinylidene groups, and the like.

The methylene group that may be substituted is, for example, a methylene group in which at least one hydrogen atom on the carbon atom is substituted with a substituent such as a hydrocarbyl group, alkoxy group, acyloxy group, alkylthio group, cyano group and a halogen atom; and typical examples thereof include ethane-1,1-diyl, propane-1,1-diyl, propane-2,2-diyl, phenylmethylene, 1-phenylethane-1,1-diyl, diphenylmethylene, dibenzylmethylene, dimethoxymethylene, diethoxymethylene, diacetoxymethylene, di(methylthio)methylene, di(ethylthio)methylene, dicyanomethylene and difluoromethylene groups, and the like.

The alkenylidene group that may be substituted is, for example, an alkenylidene group in which at least one hydrogen atom on the carbon atom is substituted with a substituent group such as a hydrocarbyl group, a cyano group or a halogen atom; and typical examples thereof include propen-1,1-diyl, 2-methylpropen-1,1-diyl, 2-phenylethen-1,1-diyl, 2,2-diphenylethen-1,1-diyl, 3-phenyl-1-propen-1,1-diyl, 2,2-dicyanoethen-1,1-diyl and 2,2-difluoroethen-1,1-diyl groups, and the like.

The carbonimidoyl group that may be substituted is, for example, a carbonimidoyl group in which the hydrogen atom on the nitrogen atom is substituted with a substituent such as the hydrocarbyl group described below; and typical examples thereof include N-methylcarbonimidoyl, N-phenylcarbonimidoyl and N-benzylcarbonimidoyl groups, and the like.

The silylene group that may be substituted is, for example, a silylene group in which at least one hydrogen atom on the silicon atom is substituted with a substituent such as a hydrocarbyl group; and typical examples thereof include dimethylsilylene, diethylsilylene, methylphenylsilylene, diphenylsilylene and dibenzylsilylene groups, and the like.

Examples of the borylene groups that may be substituted include a (2,4,6-trimethylphenyl)borylene group and the like.

In addition, when the bivalent atomic group has two or more substituents, they may bind to each other to form a ring independently. Typical examples of the rings formed include cyclopropan-1,1-diyl, cyclobutan-1,1-diyl, cyclopentan-1,1-diyl, cyclohexan-1,1-diyl, 9H-fluoren-9,9-diyl, 1,3-dioxolan-2,2-diyl, 1,3-dioxan-2,2-diyl, 1,3-dithiolan-2,2-diyl, 1,3-dithian-2,2-diyl and 9H-silafluoren-9,9-diyl groups, and the like. The formed ring may be substituted additionally with a suitable substituent, for example, a substituent described in the substituents R^{A} to R^{D} below.

In addition, preferable examples of bivalent atoms or atomic groups constituting Q also include a bivalent atomic group formed by binding in series or condensing of two to five of the bivalent atoms and atomic groups selected from the group above. Example forms of series bonds presented by names and structural formulae include as follows; an ethylene group: [—CH₂CH₂—], a cis-ethene-1,2-diyl group: [—CH═CH—], a trimethylene group: [—CH₂CH₂CH₂—], a phenylene group: [—C₆H₄—], an ethylenedioxy group: [—OCH₂CH₂O—], a trimethylenedioxy group: [—OCH₂CH₂CH₂O—], a phenylenedioxy group: [—OC₆H₄O—], a carbonyloxy group: [—O(C═O)—], a carbonyldioxy group: [—O(C═O)O—], a carbonylthio group: [—S(C═O)—], a carbonyldithio group: [—S(C═O)S—], a carbonylimino group: [—NR(C═O)—], a carbonyldiimino group: [—NR(C═O)NR—], a thiocarbonyloxy group: [—O(C═S)—], a thiocarbonyldioxy group: [—O(C═S)O—], a thiocarbonylthio group: [—S(C═S)—], a thiocarbonyldithio group: [—S(C═S)S—], a thiocarbonylimino group: [—NR(C═S)—], a thiocarbonyldiimino group: [—NR(C═S)NR—], a silylenedioxy group: [—O(SiR₂)O—], and the like. The bivalent atomic group formed by binding in series or condensation may be substituted by suitable substituent or substituens and when plural substituents exist on the atoms and/or atomic groups it may be independently bound each other to form a ring.

Examples of more preferable bivalent atoms or atomic groups constituting the group Q include an oxy group, a thio group, a sulfonyl group, an imino group that may be substituted, a methylene group that may be substituted, an alkenylidene group that may be substituted, a carbonyl group, a thiocarbonyl group and a silylene group that may be substituted, and the like.

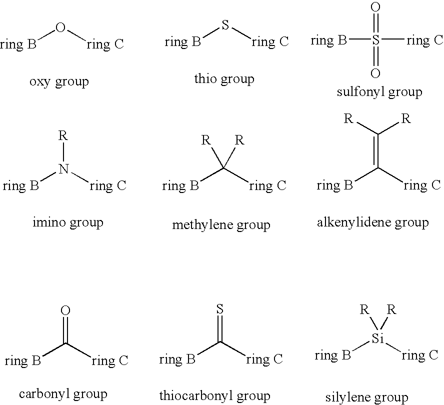

The group Z in the compound represented by General Formula (2) represents a hydrogen atom when the corresponding X is a carbon atom that can be bound with the platinum atom covalently, or a nitrogen atom that can be bound with the platinum atom covalently or a lone electron pair when the corresponding X is a nitrogen atom that can be bound with the platinum atom by coordination.

The groups R^{A}, R^{B}, R^{C}, and R^{D} in the compound of the present invention represent substituents respectively on rings A to D. Examples of the substituents include hydrocarbyl, aliphatic heterocyclic, aromatic heterocyclic, hydroxyl, alkoxy, aryloxy, aralkyloxy, heteroaryloxy, acyloxy, carbonato, acyl, carboxyl, alkoxycarbonyl, aryloxycarbonyl, aralkyloxycarbonyl, heteroaryloxycarbonyl, carbamoyl, hydroxamic acid, mercapto, alkylthio, arylthio, aralkylthio, heteroarylthio, acylthio, alkoxycarbonylthio, sulfinyl, sulfino, sulfenamoyl, sulfonyl, sulfo, sulfamoyl, amino, hydrazino, ureido, nitro, phosphino, phosphinyl, phosphinico, phosphono, silyl, boryl, and cyano groups, halogen atoms, and the like. Hereinafter, examples of these groups will be shown by the structural formulae connected to the ring. The following structural formulae represent only typical structures, and the substituents are not limited thereto. In the Formulae, R represents a hydrogen atom or an optional substituent.

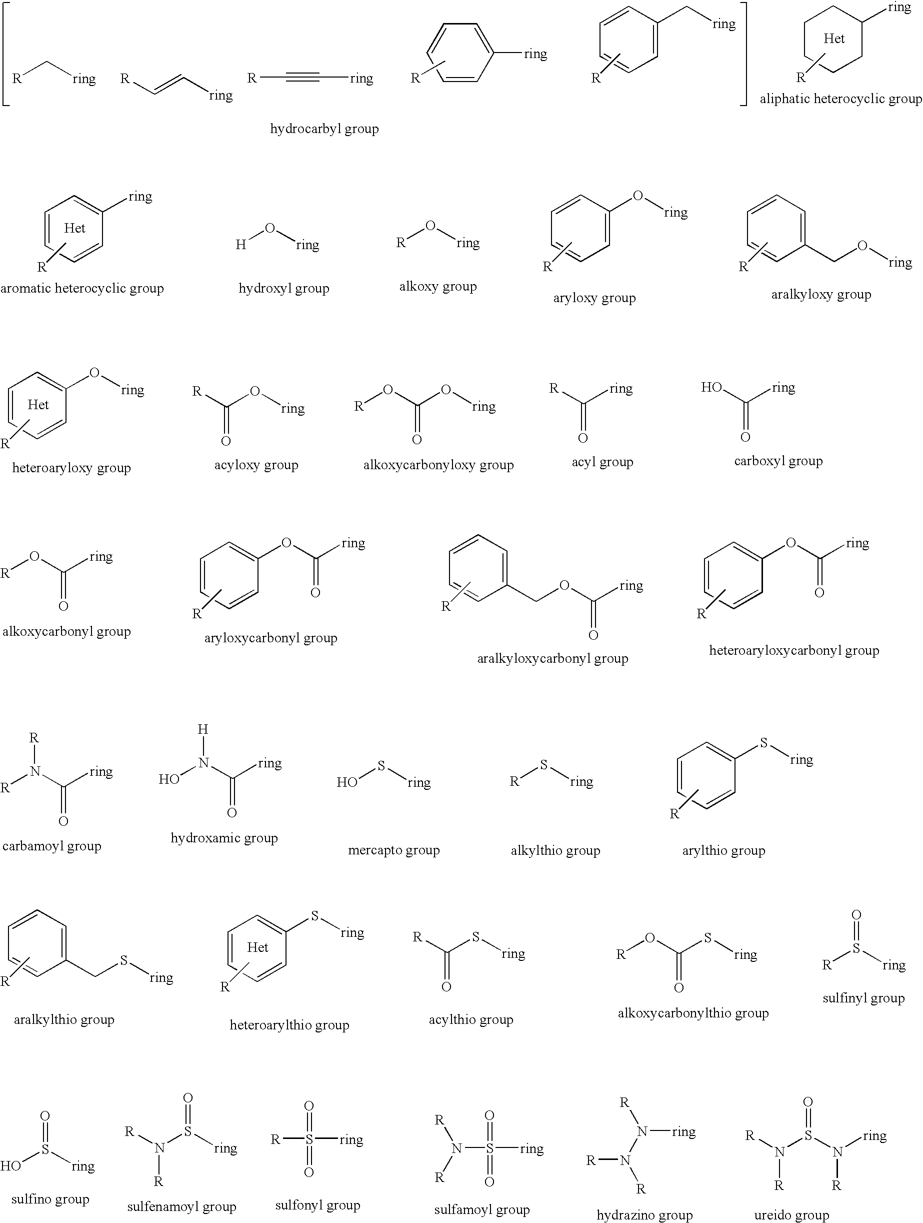

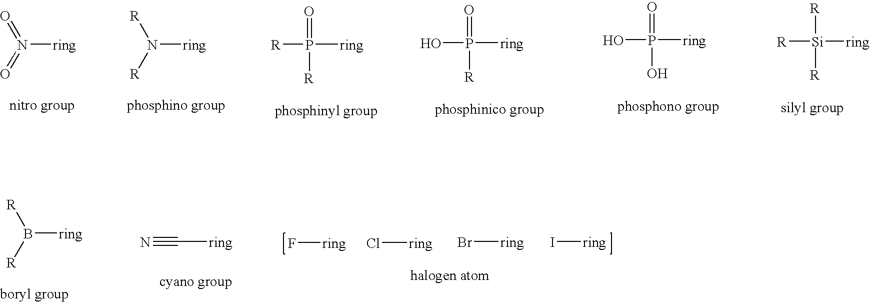

The substituents on R^{A}, R^{B}, R^{C}, and R^{D} will be described below in more detail. Examples of the hydrocarbyl groups include alkyl, alkenyl, alkynyl, aryl and aralkyl groups, and the like. Among them, the alkyl group is an straight-chain, branched, or cyclic alkyl group having, for example, 1 to 15 carbon atoms, preferably having 1 to 10 carbon atoms, and more preferably having 1 to 6 carbon atoms; and typical examples thereof include methyl, ethyl, n-propyl, 2-propyl, n-butyl, 2-butyl, isobutyl, tert-butyl, n-pentyl, 2-pentyl, tert-pentyl, 2-methylbutyl, 3-methylbutyl, 2,2-dimethylpropyl, n-hexyl, 2-hexyl, 3-hexyl, tert-hexyl, 2-methylpentyl, 3-methylpentyl, 4-methylpentyl, 2-methyl pentan-3-yl, cyclopropyl, cyclobutyl, cyclopentyl and cyclohexyl groups, and the like. The alkenyl group is a straight-chain or branched alkenyl group having, for example, 2 to 15 carbon atoms, preferably having 2 to 10 carbon atoms, and more preferably having 2 to 6 carbon atoms; and typical examples thereof include ethenyl, propenyl, 1-butenyl, pentenyl and hexenyl groups, and the like. The alkynyl group is a straight-chain or branched alkynyl group having, for example, 2 to 15 carbon atoms, preferably having 2 to 10 carbon atoms, and more preferably having 2 to 6 carbon atoms; and typical examples thereof include ethynyl, 1-propynyl, 2-propynyl, 1-butynyl, 3-butynyl, pentynyl and hexynyl groups, and the like. The aryl group is an aryl group having, for example, 6 to 14 carbon atoms; and typical examples thereof include phenyl, naphthyl, anthryl, phenanthrenyl, chrysenyl, pyrenyl and biphenyl groups, and the like. The aralkyl group is a group in which at least one hydrogen atom of the alkyl group is substituted with the aryl group above, preferably an aralkyl group having, for example, 7 to 13 carbon atoms; and typical examples thereof include benzyl, 2-phenylethyl, 1-phenylpropyl and 3-naphthylpropyl groups, and the like.

Examples of the aliphatic heterocyclic group include five- to eight-membered, preferably five- or six-membered, monocyclic aliphatic heterocyclic and polycyclic or fused aliphatic heterocyclic groups, for example, having 2 to 14 carbon atoms and containing at least one, preferably one to three, heteroatom such as nitrogen, oxygen, or sulfur. Typical examples of the aliphatic heterocyclic groups include pyrrolidyl-2-one, piperidino, piperadinyl, morpholino, tetrahydrofuryl, tetrahydropyranyl and tetrahydrothienyl groups, and the like.

Examples of the aromatic heterocyclic group include five- to eight-membered, preferably five- or six-membered, monocyclic hetero-aryl and polycyclic or fused hetero-aryl groups having, for example, 2 to 15 carbon atoms and containing at least one, preferably one to three, heteroatom such as nitrogen, oxygen, or sulfur; and typical examples thereof include furyl, thienyl, pyridyl, pyrimidyl, pyradyl, pyridazyl, pyrazolyl, imidazolyl, oxazolyl, thiazolyl, benzofuryl, benzothienyl, quinolyl, isoquinolyl, quinoxalyl, phthalazyl, quinazolyl, naphthylidyl, cinnolyl, benzimidazolyl, benzoxazolyl and benzothiazolyl groups, and the like.

The alkoxy group is a straight-chain, branched, or cyclic alkoxy group having, for example, 1 to 6 carbon atoms; and typical examples thereof include methoxy, ethoxy, n-propoxy, 2-propoxy, n-butoxy, 2-butoxy, isobutoxy, tert-butoxy, n-pentyloxy, 2-methylbutoxy, 3-methylbutoxy, 2,2-dimethylpropyloxy, n-hexyloxy, 2-methylpentyloxy, 3-methylpentyloxy, 4-methylpentyloxy, 5-methylpentyloxy and cyclohexyloxy groups, and the like.

The aryloxy group is an aryloxy group having, for example, 6 to 14 carbon atoms; and typical examples thereof include phenyloxy, naphthyloxy and anthryloxy groups, and the like.

The aralkyloxy group is an aralkyloxy group having, for example, 7 to 12 carbon atoms; and typical examples thereof include benzyloxy, 2-phenylethoxy, 1-phenylpropoxy, 2-phenylpropoxy, 3-phenylpropoxy, 1-phenylbutoxy, 2-phenylbutoxy, 3-phenylbutoxy, 4-phenylbutoxy, 1-phenylpentyloxy, 2-phenylpentyloxy, 3-phenylpentyloxy, 4-phenylpentyloxy, 5-phenylpentyloxy, 1-phenylhexyloxy, 2-phenylhexyloxy, 3-phenylhexyloxy, 4-phenylhexyloxy, 5-phenylhexyloxy and 6-phenylhexyloxy groups, and the like.

The heteroaryloxy group is a heteroaryloxy group, for example, having 2 to 14 carbon atoms and containing at least one, preferably one to three, heteroatom such as nitrogen, oxygen, or sulfur; and typical examples thereof include 2-pyridyloxy, 2-pyrazyloxy, 2-pyrimidyloxy and 2-quinolyloxy groups, and the like.

The acyloxy group is an acyloxy group having, for example, 2 to 18 carbon atoms and derived from carboxylic acid; and typical examples thereof include acetoxy, propionyloxy, acryloyloxy, butyryloxy, pivaloyloxy, pentanoyloxy, hexanoyloxy, lauroyloxy, stearoyloxy and benzoyloxy groups, and the like.

The alkoxycarbonyloxy group, is a straight-chain, branched, or cyclic alkoxycarbonyloxy group having, for example, 2 to 19 carbon atoms; and typical examples thereof include methoxycarbonyloxy, ethoxycarbonyloxy, n-propoxycarbonyloxy, 2-propoxycarbonyloxy, n-butoxycarbonyloxy, tert-butoxycarbonyloxy, pentyloxycarbonyloxy, hexyloxycarbonyloxy, 2-ethylhexyloxycarbonyloxy, lauryloxycarbonyloxy, stearyloxycarbonyloxy and cyclohexyloxycarbonyloxy groups, and the like.

The acyl group is a straight-chain or branched acyl group having, for example, 1 to 18 carbon atoms and derived from a carboxylic acid such as a fatty carboxylic acid or an aromatic carboxylic acid; and typical examples thereof include formyl, acetyl, propionyl, acryloyl, butyryl, pivaloyl, pentanoyl, hexanoyl, lauroyl, stearoyl and benzoyl groups, and the like.

The alkoxycarbonyl group is a straight-chain, branched, or cyclic alkoxycarbonyl group having, for example, 2 to 19 carbon atoms; and typical examples thereof include methoxycarbonyl, ethoxycarbonyl, n-propoxycarbonyl, 2-propoxycarbonyl, n-butoxycarbonyl, tert-butoxycarbonyl, pentyloxycarbonyl, hexyloxycarbonyl, 2-ethylhexyloxycarbonyl, lauryloxycarbonyl, stearyloxycarbonyl and cyclohexyloxycarbonyl groups, and the like.

The aryloxycarbonyl group is an aryloxycarbonyl group having, for example, 7 to 20 carbon atoms; and typical examples thereof include phenoxycarbonyl and naphthyloxycarbonyl groups, and the like.

The aralkyloxycarbonyl group is an aralkyloxycarbonyl group having, for example, 8 to 15 carbon atoms; and typical examples thereof include benzyloxycarbonyl, phenylethoxycarbonyl and 9-fluorenylmethyloxycarbonyl groups, and the like.

The heteroaryloxycarbonyl group is a heteroaryloxy group having, for example, 3 to 15 carbon atoms and containing at least one, preferably one to three, heteroatom such as a nitrogen, oxygen, or sulfur atom; and typical examples thereof include 2-pyridyloxycarbonyl, 2-pyrazyloxycarbonyl, 2-pyrimidyloxycarbonyl and 2-quinolyloxycarbonyl groups, and the like.

The carbamoyl group is, for example, an unsubstituted carbamoyl group or a carbamoyl group, one or two hydrogen atoms on the nitrogen atom of which are substituted with a substituent group such as the hydrocarbyl group described above; and typical examples thereof include N-methylcarbamoyl, N,N-diethylcarbamoyl and N-phenylcarbamoyl groups, and the like.

The alkylthio group is a straight-chain, branched, or cyclic alkylthio group having, for example, 1 to 6 carbon atoms; and typical examples thereof include methylthio, ethylthio, n-propylthio, 2-propylthio, n-butylthio, 2-butylthio, isobutylthio, tert-butylthio, pentylthio, hexylthio and cyclohexylthio groups, and the like.

The arylthio group is an arylthio group having, for example, 6 to 14 carbon atoms; and typical examples thereof include phenylthio and naphthylthio groups and the like. The aralkylthio group is an aralkylthio group having, for example, 7 to 12 carbon atoms; and typical examples thereof include benzylthio and 2-phenethylthio groups and the like.

The heteroarylthio group is a heteroarylthio group having, for example, 2 to 14 carbon atoms and containing at least one, preferably one to three, heteroatom such as a nitrogen, oxygen, or sulfur atom; and typical examples thereof include 4-pyridylthio, 2-benzimidazolylthio, 2-benzoxazolylthio and 2-benzothiazolylthio groups, and the like.

The acylthio group is an acylthio group having, for example, 2 to 18 carbon atoms and derived from a thiocarboxylic acid; and typical examples thereof include acetylthio, propionylthio, acrylthio, butyrylthio, pivaloylthio, pentanoylthio, hexanoylthio, lauroylthio, stearoylthio and benzoylthio groups, and the like.

The alkoxycarbonylthio group is a straight-chain, branched, or cyclic alkoxycarbonylthio group having, for example, 2 to 19 carbon atoms; and typical examples thereof include methoxycarbonylthio, ethoxycarbonylthio, n-propoxycarbonylthio, 2-propoxycarbonylthio, n-butoxycarbonylthio, tert-butoxycarbonylthio, pentyloxycarbonylthio, hexyloxycarbonylthio, 2-ethylhexyloxycarbonylthio, lauryloxycarbonylthio, stearyloxycarbonylthio and cyclohexyloxycarbonylthio groups, and the like.

The sulfinyl group is, for example, a sulfinyl group, of which the hydrogen atom on the sulfur atom is substituted with a substituent such as the hydrocarbyl group described above; and typical examples thereof include methanesulfinyl, benzenesulfinyl and p-toluenesulfinyl groups, and the like.

The sulfenamoyl group is, for example, an unsubstituted sulfenamoyl group or a sulfenamoyl group, of which the hydrogen atom on the nitrogen atom is substituted with a substituent such as the hydrocarbyl group described above; and typical examples thereof include N-methylsulfenamoyl, N,N-diethylsulfenamoyl and N-phenylsulfenamoyl groups, and the like.

The sulfonyl group is, for example, a sulfonyl group, of which the hydrogen atom on the sulfur atom is substituted with a substituent such as the hydrocarbyl group described above; and typical examples thereof include methanesulfonyl, benzenesulfonyl and p-toluenesulfonyl groups, and the like.

The sulfamoyl group is, for example, an unsubstituted sulfamoyl group or a sulfamoyl group, of which the hydrogen atom on the nitrogen atom is substituted with a substituent such as the hydrocarbyl group described above; and typical examples thereof include N-methylsulfamoyl, N,N-diethylsulfamoyl and N-phenylsulfamoyl groups, and the like.

The amino group is, for example, an unsubstituted amino group or an amino group, of which the hydrogen atom on the nitrogen atom is substituted with a substituent such as an amino-protecting group. For example, any one of the protecting groups described in Non-patent Document 2 may be used as the amino-protecting group, and typical examples thereof include the alkyl, aryl, aralkyl, acyl, alkoxycarbonyl, aryloxycarbonyl, aralkyloxycarbonyl and sulfonyl groups described above, and the like.

Typical examples of the alkyl group-substituted amino group, i.e., alkylamino group, include mono- or di-alkylamino groups such as N-methylamino, N,N-dimethylamino, N,N-diethylamino, N,N-diisopropylamino and N-cyclohexylamino groups, and the like.

Typical examples of the aryl group-substituted amino group, i.e., arylamino group include mono- or di-arylamino groups such as N-phenylamino, N,N-diphenylamino, N-naphthylamino and N-naphthyl-N-phenylamino groups.

Typical examples of the aralkyl group-substituted amino group, i.e., aralkylamino group, include mono- or di-aralkylamino groups such as N-benzylamino and N,N-dibenzylamino groups.

Typical examples of the acyl group-substituted amino group, i.e., acylamino group, include formylamino, acetylamino, propionylamino, acryloylamino, pivaloylamino, pentanoylamino, hexanoylamino and benzoylamino groups, and the like.

Typical examples of the alkoxycarbonyl group-substituted amino group, i.e., alkoxycarbonylamino group, include methoxycarbonylamino, ethoxycarbonylamino, n-propoxycarbonylamino, n-butoxycarbonylamino, tert-butoxycarbonylamino, pentyloxycarbonylamino and hexyloxycarbonylamino groups, and the like.

Typical examples of the aryloxycarbonyl group-substituted amino group, i.e., aryloxycarbonylamino group, include phenoxycarbonylamino and naphthyloxycarbonylamino groups, and the like.

Typical examples of the aralkyloxycarbonyl group-substituted amino group, i.e., aralkyloxycarbonylamino group include a benzyloxycarbonylamino group and the like.

Typical examples of the sulfonyl group-substituted amino group, i.e., sulfonylamino group, include methanesulfonylamino and p-toluenesulfonylamino groups, and the like.

The hydrazino group is, for example, an unsubstituted hydrazino group and a hydrazino group, of which at least one hydrogen atom on the nitrogen atom is substituted with a substituent such as the hydrocarbyl group described above; and typical examples thereof include 2-methylhydrazino, 2,2-dimethylhydrazino, 1,2,2-trimethylhydrazino, 2-phenylhydrazino and 2,2-diphenylhydrazino groups, and the like.

The ureido group is, for example, an unsubstituted ureido group or a ureido group, of which at least one hydrogen atom on the nitrogen atom is substituted with a substituent such as the hydrocarbyl group described above; and typical examples thereof include 3-methylureido, 1,3,3-trimethylureido and 3,3-diphenylureido groups, and the like.

The phosphino group is, for example, a phosphino group, of which two hydrogen atoms on the phosphorus atom are substituted with a substituent such as the hydrocarbyl group described above; and typical examples thereof include dimethylphosphino, diphenylphosphino, di(2-furyl)phosphino and dibenzylphosphino groups, and the like.

The phosphinyl group is, for example, a phosphinyl group, of which two hydrogen atoms on the phosphorus atom are substituted with a substituent such as the hydrocarbyl group described above; and typical examples thereof include dimethylphosphinyl and diphenylphosphinyl groups, and the like.

The phosphinico group is, for example, an unsubstituted phosphinico group or a phosphinico group, of which the hydrogen atom on the oxygen atom is substituted with a substituent such as the hydrocarbyl group described above; and typical examples thereof include methylphosphinico, ethylphosphinico, phenylphosphinico and benzylphosphinico groups, and the like.

The phosphono group is, for example, an unsubstituted phosphono group or a phosphono group, of which the hydrogen atom on the oxygen atom is substituted with a substituent such as the hydrocarbyl group described above; and typical examples thereof include dimethylphosphono, diethylphosphono, phenylphosphono, diphenylphosphono and dibenzylphosphono groups, and the like.

The silyl group is, for example, a silyl group, of which the hydrogen atom on the silicon atom is substituted with a substituent such as the hydrocarbyl group described above; and typical examples thereof include trimethylsilyl, triisopropylsilyl, tert-butyldimethylsilyl, tert-butyldiphenylsilyl and triphenylsilyl groups, and the like.

The boryl group is, for example, a boryl group, of which the two hydrogen atoms on the boron atom are substituted with a substituent such as the hydrocarbyl group described above; and typical examples thereof include a bis(2,4,6-trimethylphenyl) boryl group and the like.

Examples of the halogen atoms include fluorine, chlorine, bromine and iodine atoms, and the like.

When there are two or more substituents on the same ring, these substituents may be bound to each other independently to form a fused ring. In addition, when neighboring rings have respectively one or more substituents, these substituents may be bound each other independently to form a fused ring. Typical examples of the fused rings formed by the substituents on rings A and B and by the substituents on rings C and D are shown below:

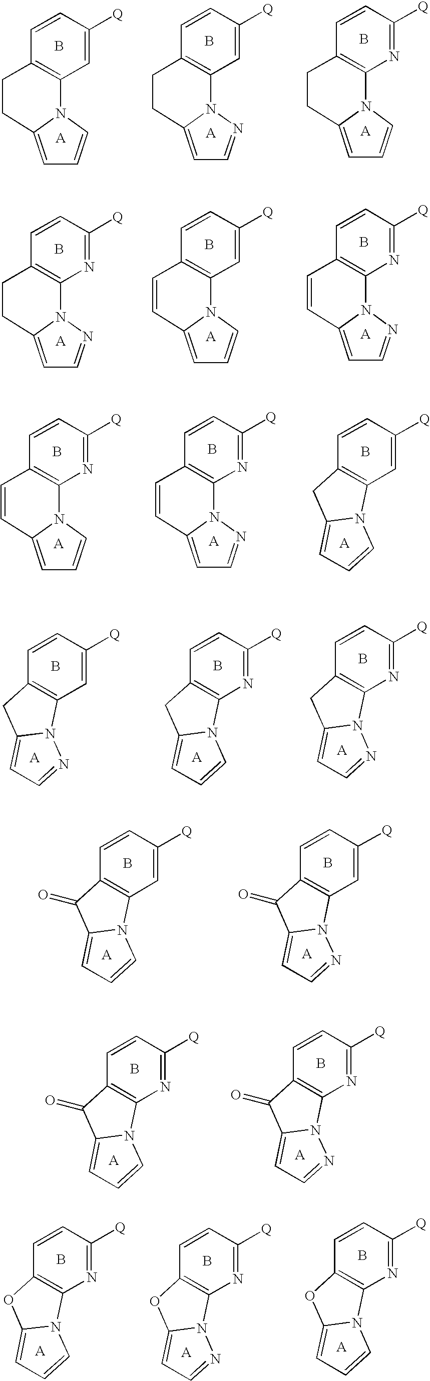

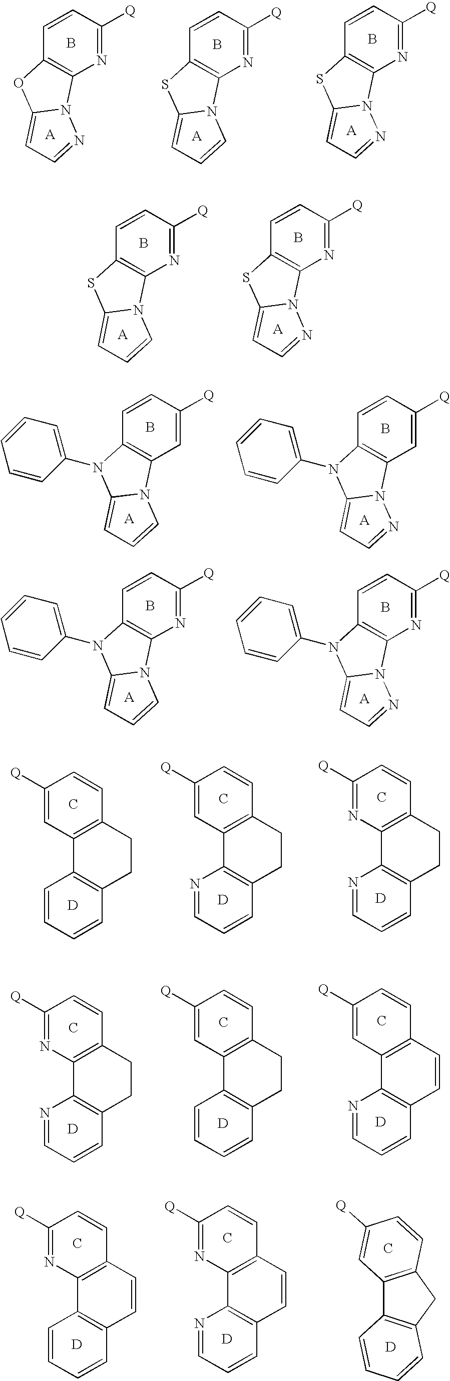

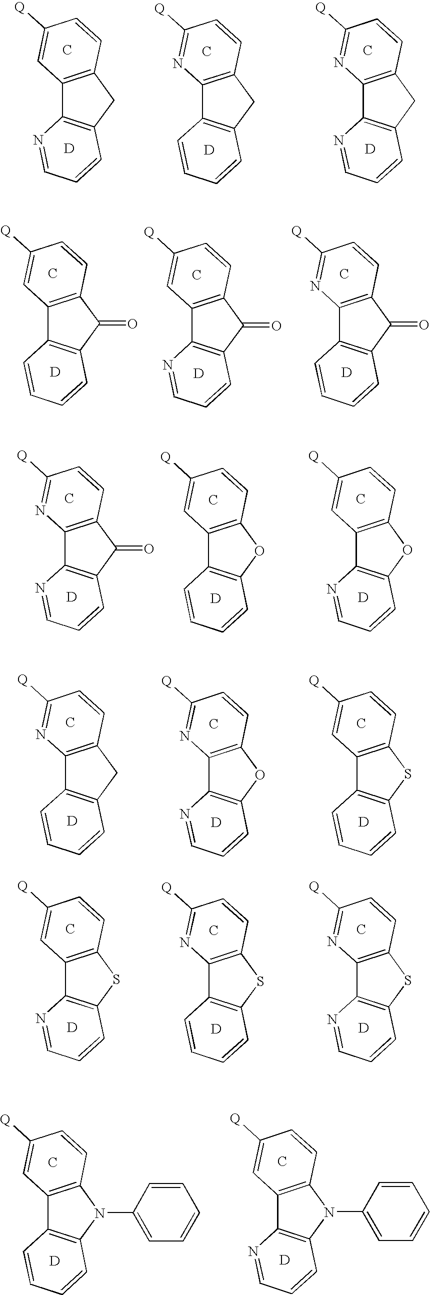

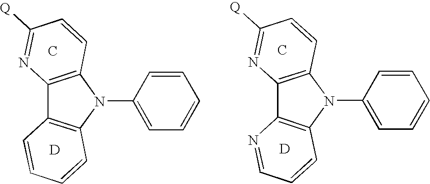

Hereinafter, typical examples of the platinum complex represented by General Formula (1) of the present invention are listed below, but the present invention is not restricted thereby.

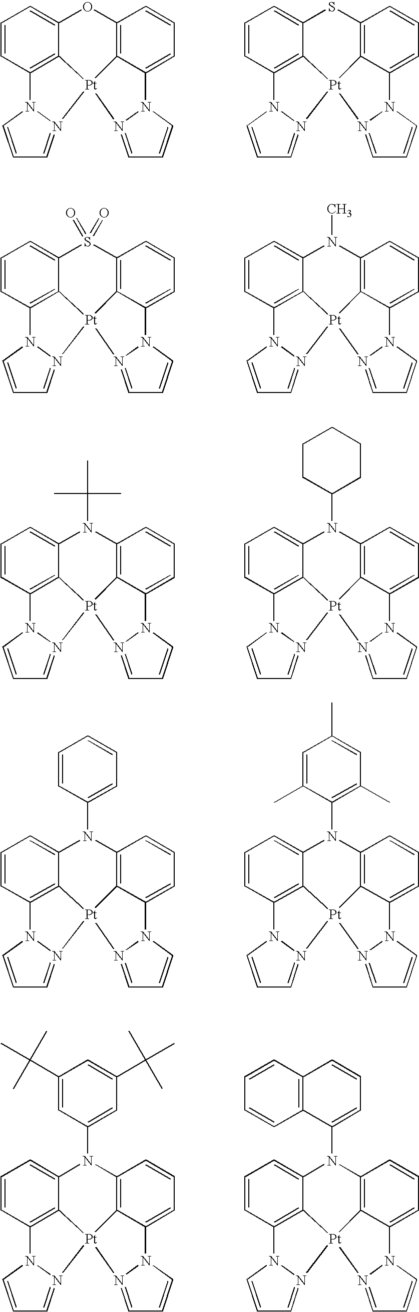

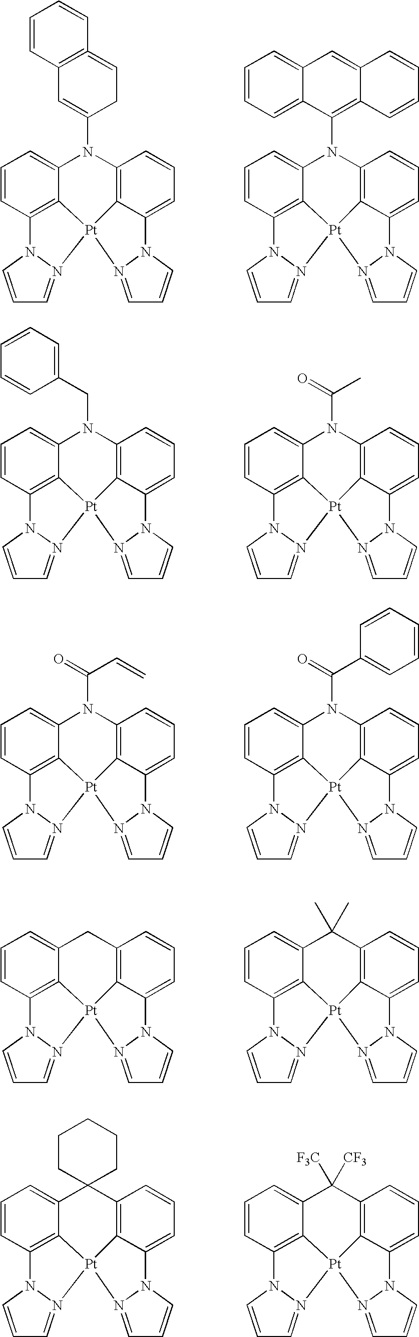

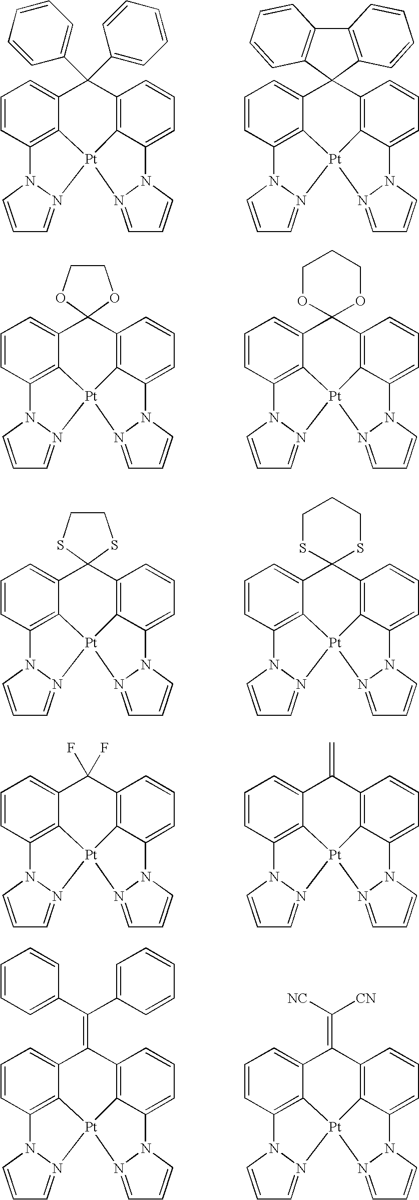

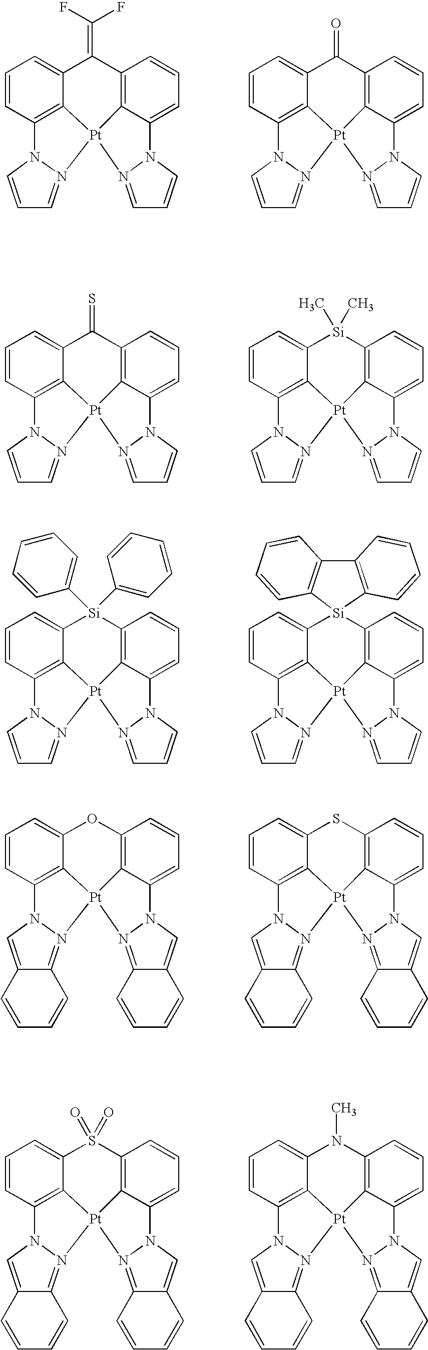

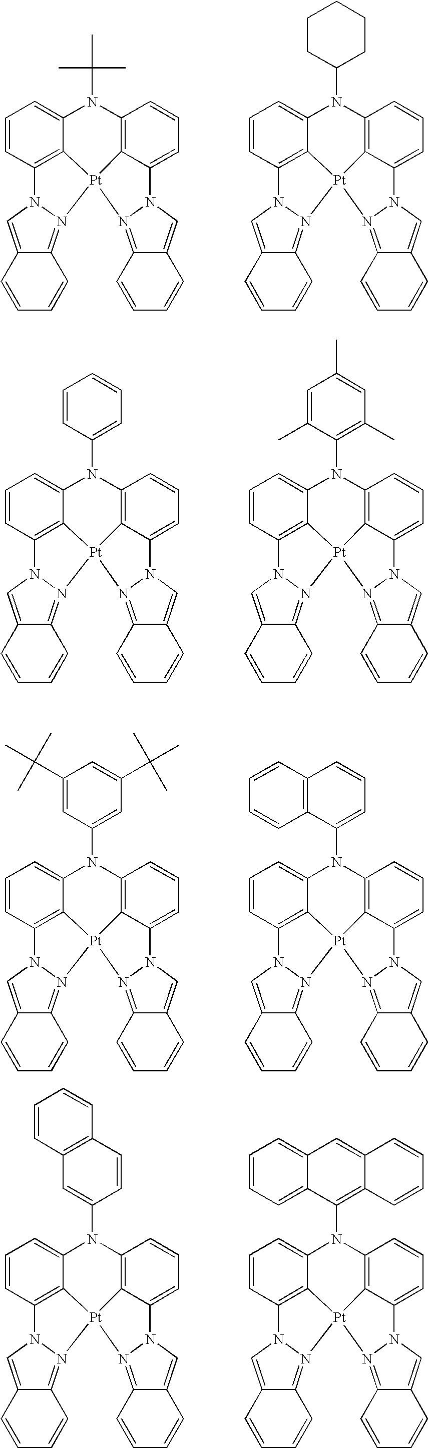

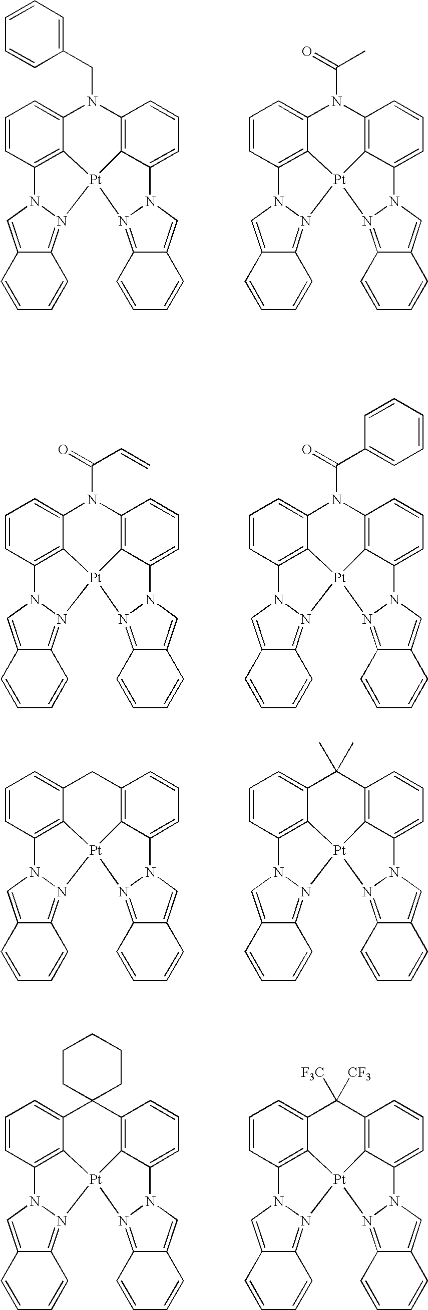

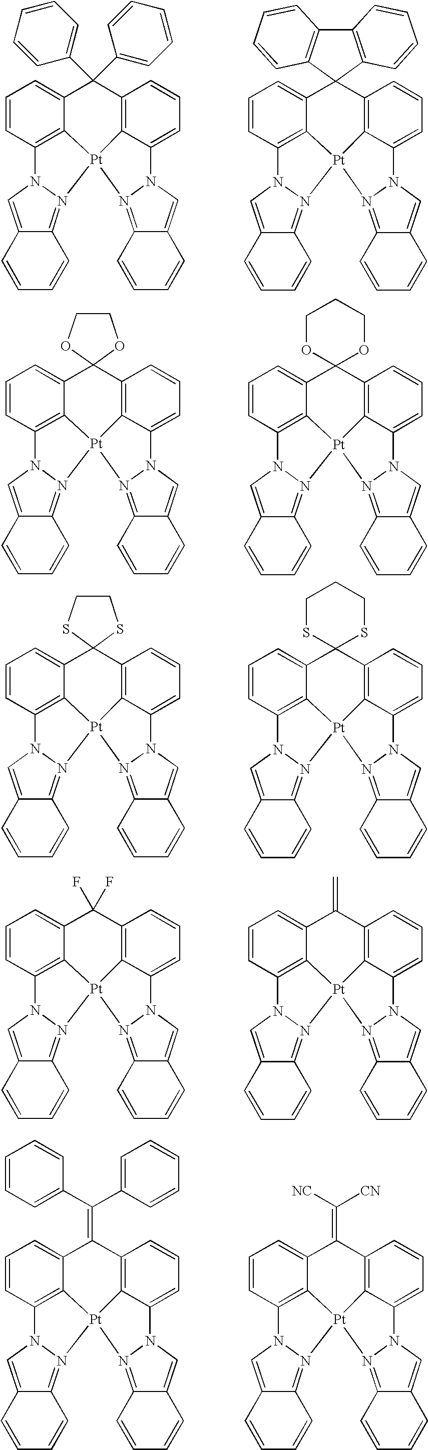

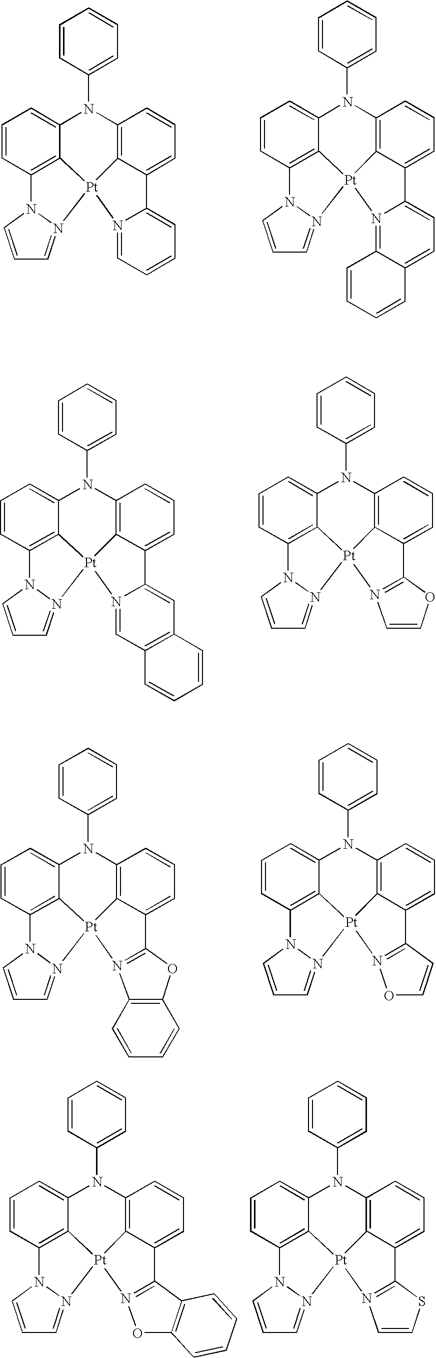

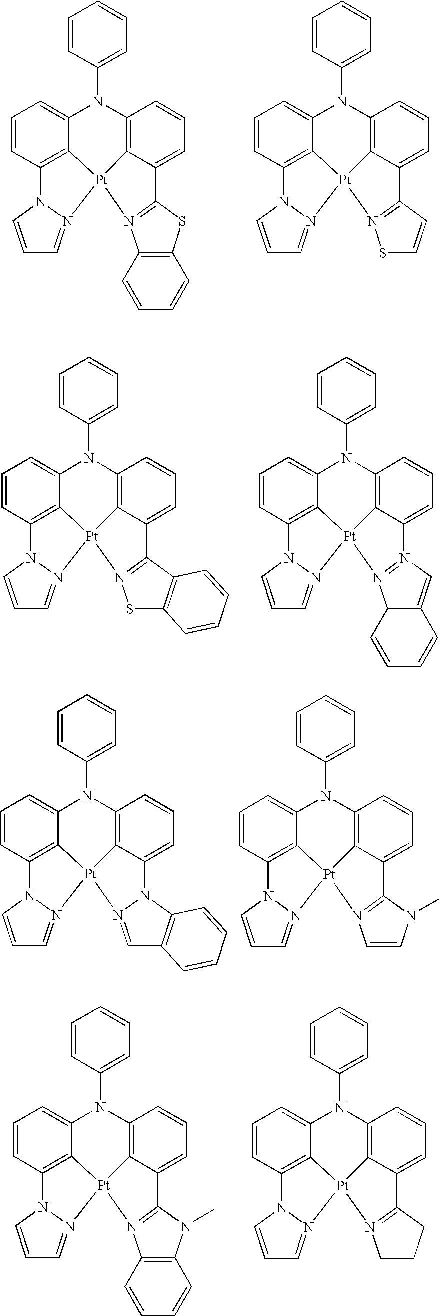

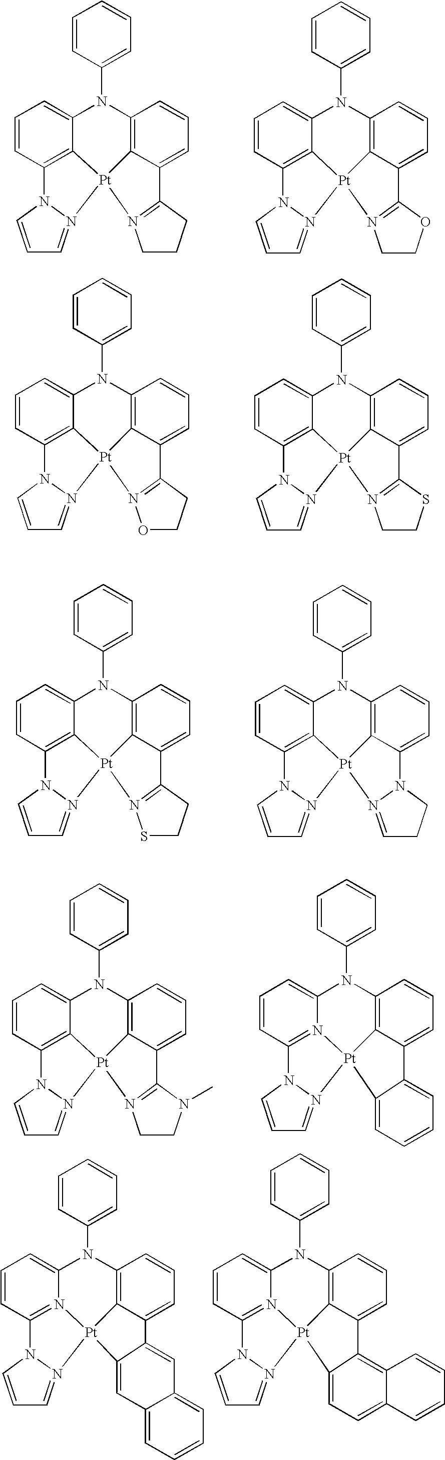

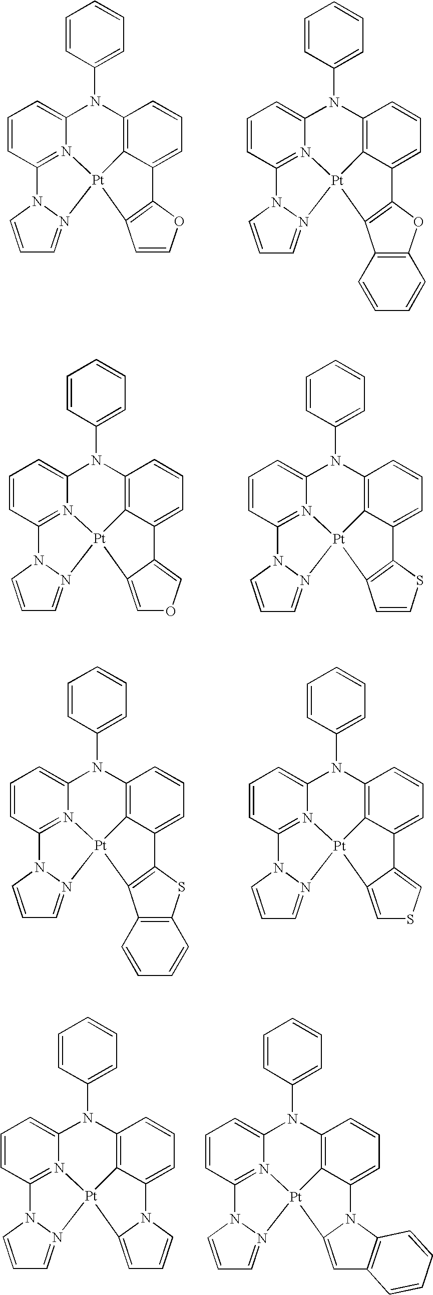

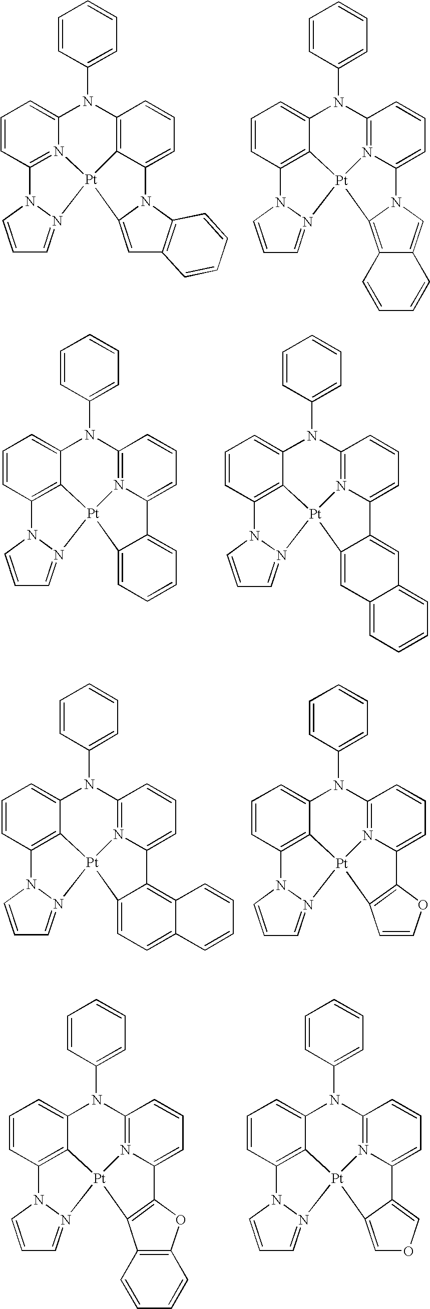

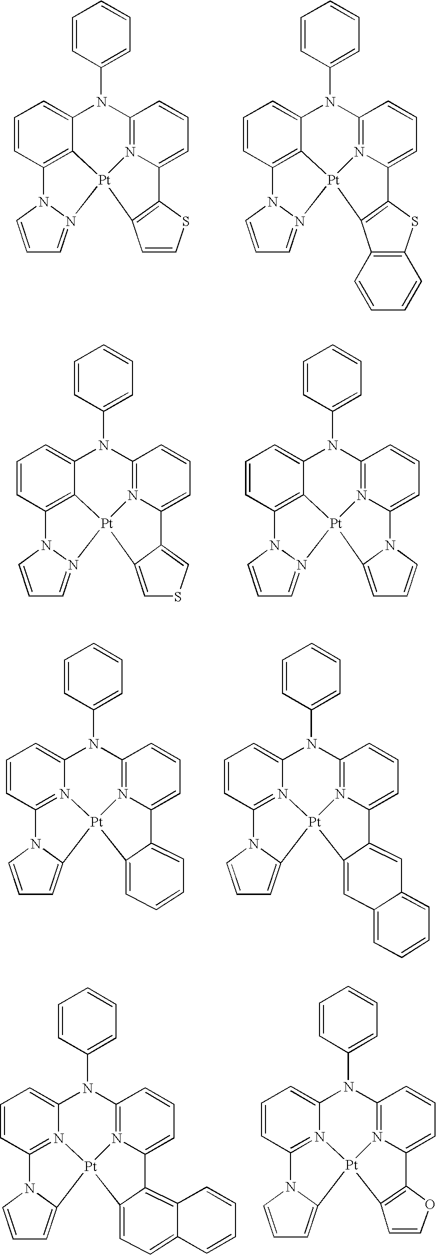

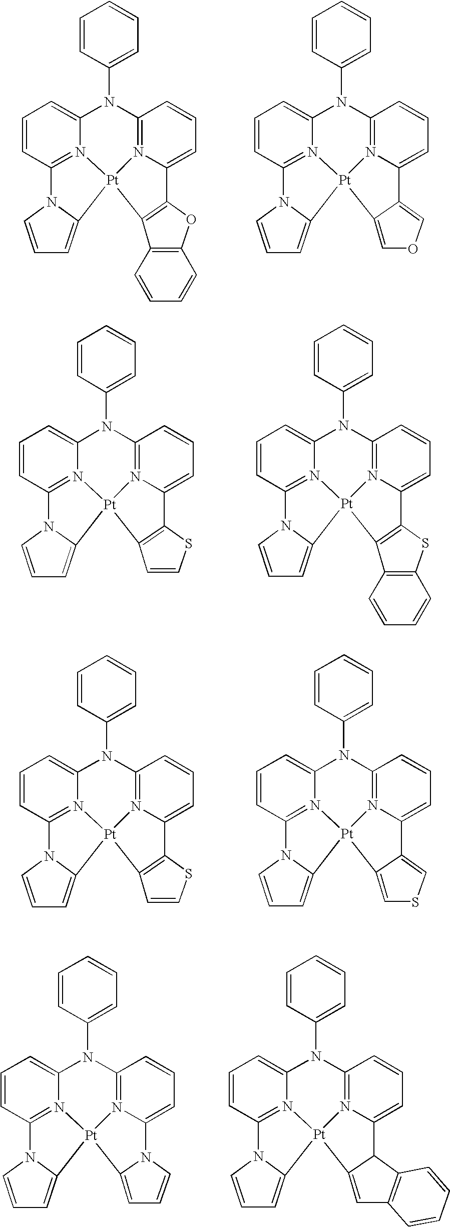

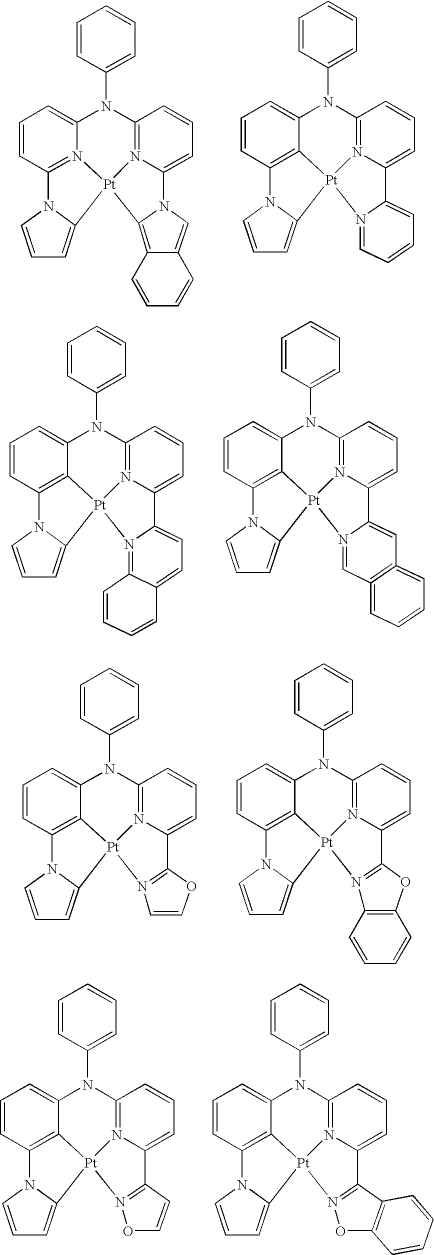

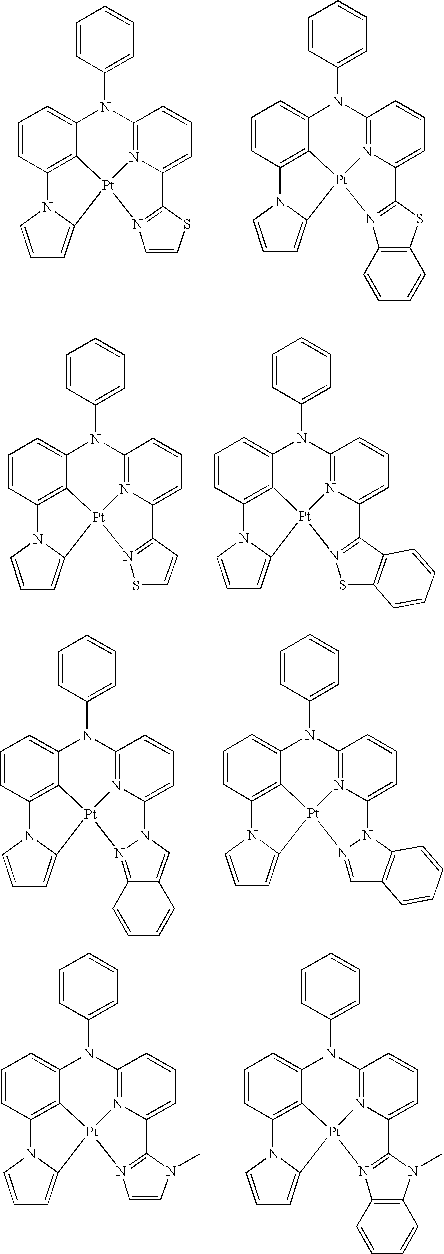

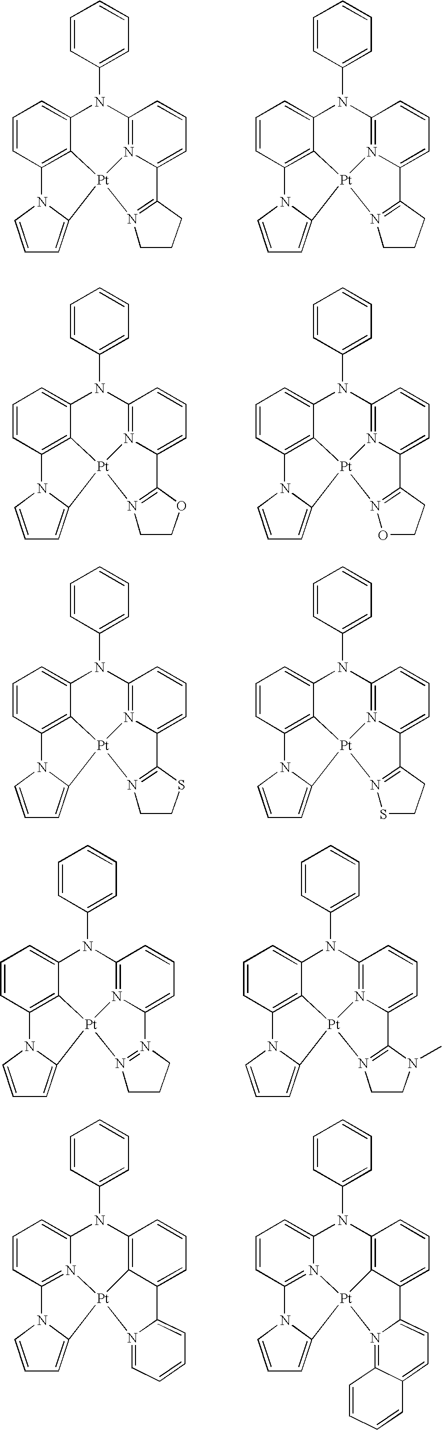

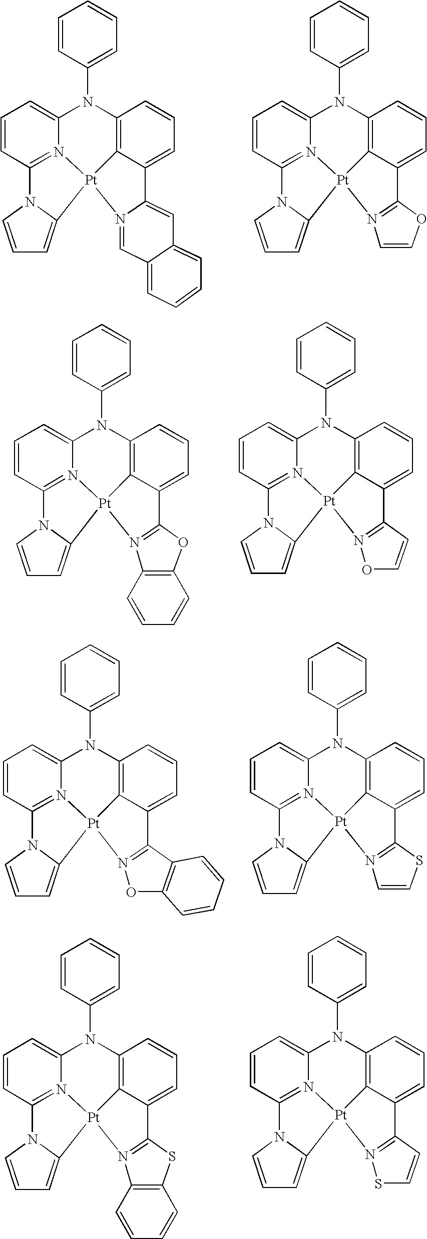

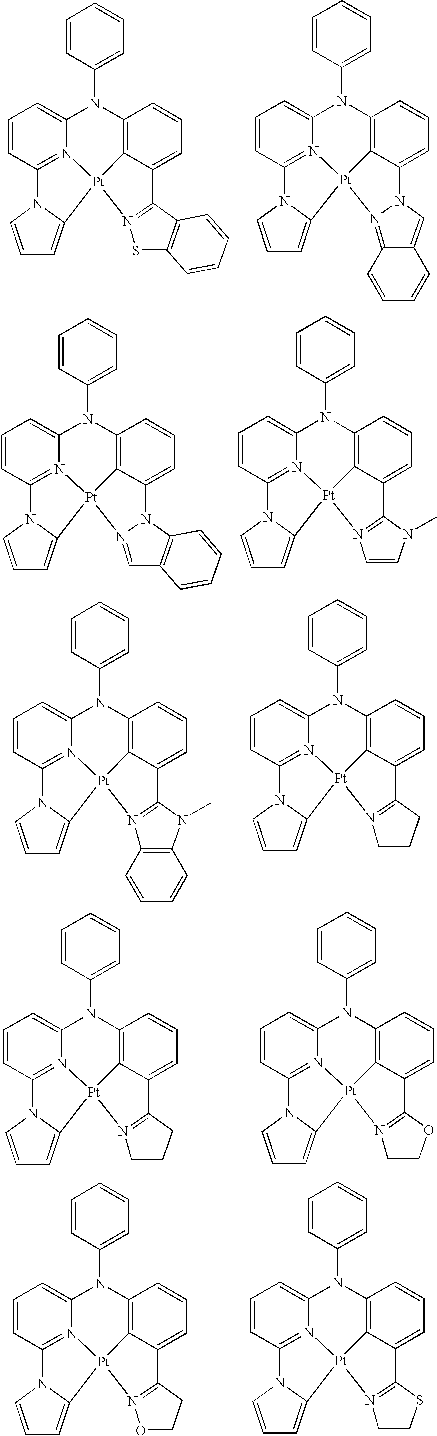

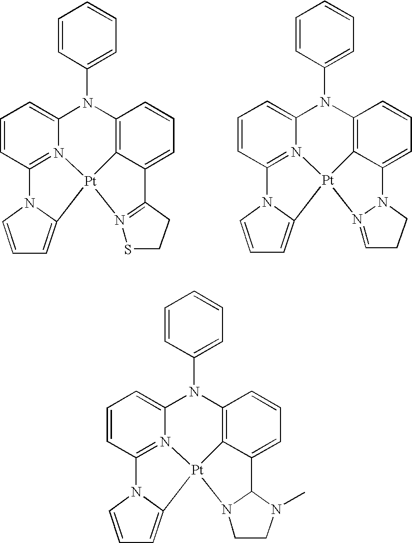

Hereinafter, the method of producing the platinum complex of the present invention will be described.

The compound represented by General Formula (1) can be prepared easily in the reaction of a platinum complex precursor with the compound represented by General Formula (2) as shown in the following Scheme 1: 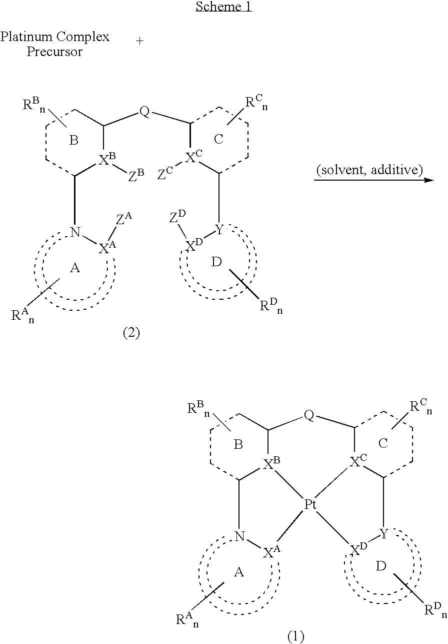
wherein the compounds represented by General Formulae (1) and (2) are the same as those described above.

Hereinafter, the compound represented by General Formula (2) will be referred to simply as “the tetradentate ligand of the present invention”.

Both inorganic and organic platinum complexes may be used favorably as the platinum complex precursor used in the production method according to the present invention. Favorable examples of the inorganic platinum compounds include platinum halides such as platinum chloride, platinum bromide and platinum iodide; and haloplatinic acid salts such as sodium chloroplatinate, potassium chloroplatinate, potassium bromoplatinate and potassium iodoplatinate. Platinum chloride and potassium chloroplatinate are used more favorably, because of the easiness in procurement.

The organic platinum complex is preferably an organic platinum complex having a monodentate or bidentate ligand from a viewpoint of chelating effect. Typical examples thereof include platinum olefin complexes such as di-μ-chloro-dichloroethylenediplatinum, dichloro(η-1,5-hexadiene)platinum, dichloro(η-1,5-cyclooctadiene)platinum, (η-bicyclo[2,2,1]hepta-2,5-diene)dichloroplatinum and bis(η-1,5-cyclooctadiene) platinum; platinum amine complexes such as cis-/trans-bis(ammine)dichloroplatinum and dichloro(ethylenediammine)platinum; platinum nitrogen-containing heterocyclic ring complexes such as cis-/trans-bis(pyridinato)dichloroplatinum and (2,2′-bipyridinato) dichloroplatinum; platinum nitrile complexes such as cis-bis(benzonitrile)dichloroplatinum and cis-/trans-bis(acetonitrile)dichloroplatinum; platinum phosphine complexes such as cis-/trans-bis(tributylphosphine)dichloroplatinum, cis-/trans-bis(triphenylphosphine)dichloroplatinum, dichloro[ethanebis(diphenylphosphine)]platinum and tetrakis(triphenylphosphine)platinum; platinum sulfur-containing compound complexes such as cis-bis(tetrahydrothiophene)dichloroplatinum; and the like.

More preferable examples of the organic platinum complexes include platinum olefin complexes such as dichloro(η-1,5-hexadiene)platinum and dichloro(η-1,5-cyclooctadiene)platinum; platinum nitrile complex such as cis-bis(benzonitrile)dichloroplatinum and cis-/trans-bis(acetonitrile)dichloroplatinum; and the like.

The organic platinum complexes above may be used in the complexation after preparation and isolation, or alternatively, in the so-called one-pot reaction thereof with the tetradentate ligand of the present invention, without isolation after preparation from an inorganic platinum compound. Specifically, cis-bis(benzonitrile)dichloroplatinum, for example, is prepared from platinum chloride and benzonitrile in a system; then a tetradentate ligand of the present invention and other additives if needed are added thereto; and the mixture is allowed to react in the benzonitrile solvent.

The amount of the tetradentate ligand of the present invention used is normally 0.5 to 20 equivalents, preferably 0.8 to 10 equivalents, and more preferably 1.0 to 2.0 equivalents to the amount of the platinum complex precursor.

The platinum complex may be prepared in the absence of a solvent, and is preferably prepared in the presence of a solvent. Typical examples of the preferable solvents include aliphatic hydrocarbons such as pentane, hexane, heptane, octane, decane, dodecane, undecane, cyclohexane and decalin; halogenated aliphatic hydrocarbons such as dichloromethane, 1,2-dichloroethane, chloroform and carbon tetrachloride; aromatic hydrocarbons such as benzene, toluene, xylene, mesitylene, p-cymene and diisopropylbenzene; halogenated aromatic hydrocarbons such as chlorobenzene and o-dichlorobenzene; alcohols such as methanol, ethanol, 2-propanol, n-butanol and 2-ethoxyethanol; polyvalent alcohols such as ethylene glycol, propylene glycol, 1,2-propanediol and glycerol; ethers such as diethyl ether, diisopropyl ether, tert-butyl methyl ether, cyclopentyl methyl ether, dimethoxy ethane, ethylene glycol diethyl ether, tetrahydrofuran and 1,4-dioxane; carboxylic acids such as acetic acid and propionic acid; esters such as methyl acetate, ethyl acetate, n-butyl acetate and methyl propionate; ketones such as acetone, methyl ethyl ketone, methyl isobutyl ketone and cyclohexanone; amines such as triethylamine, aniline and phenethylamine; amides such as formamide, N,N-dimethylformamide and N,N-dimethylacetamide; nitriles such as acetonitrile, malononitrile and benzonitrile; sulfoxides such as dimethyl sulfoxide; water; and the like. These solvents may be used alone or in combination of two or more thereof if needed.

Typical examples of more preferable solvents include aliphatic hydrocarbons such as decane, dodecane, undecane and decalin; aromatic hydrocarbons such as toluene, xylene, mesitylene, p-cymene and diisopropylbenzene; alcohols such as n-butanol and 2-ethoxyethanol; polyvalent alcohols such as ethylene glycol, propylene glycol, 1,2-propanediol and glycerol; ethers such as ethylene glycol diethyl ether, tetrahydrofuran and 1,4-dioxane; carboxylic acids such as acetic acid and propionic acid; esters such as n-butyl acetate and methyl propionate; amides such as N,N-dimethylformamide and N,N-dimethylacetamide; nitriles such as benzonitrile; sulfoxides such as dimethyl sulfoxide; water; and the like. These solvents may be used alone or in combination of two or more thereof if needed.

The amount of the solvent used is not particularly limited, if the reaction proceeds sufficiently, and is properly selected in the range of larger by 1 to 500 times, preferably by 5 to 200 times, and more preferably by 10 to 100 times by volume to the amount of the platinum complex precursor.

The platinum complex may be prepared in the presence of additives added as needed. One of the favorable additives is a base. The bases include, for example, inorganic and organic bases. Favorable examples of the inorganic bases include alkali metal hydroxides such as lithium hydroxide, sodium hydroxide and potassium hydroxide; alkali metal carbonate salts such as lithium carbonate, sodium carbonate and potassium carbonate; alkali metal bicarbonates such as sodium bicarbonate and potassium bicarbonate; and metal hydrides such as sodium hydride. Favorable examples of the organic base include alkali metal alkoxides such as lithium methoxide, sodium methoxide, potassium methoxide, sodium ethoxide, potassium ethoxide, sodium tert-butoxide and potassium tert-butoxide; amines such as triethylamine, diisopropylethylamine, N,N-dimethylaniline, piperidine, pyridine, 4-dimethylaminopyridine, 1,5-diazabicyclo[4.3.0]nona-5-ene, 1,8-diazabicyclo[5.4.0]undeca-7-ene, tri-n-butylamine and N-methylmorpholine; organic alkali metal compounds such as n-butyllithium, tert-butyllithium and phenyllithium; Grignard reagents such as butylmagnesium chloride, phenylmagnesium bromide and methylmagnesium iodide; and the like.

When a base is used as an additive, the amount thereof is properly selected in the range of normally 1 to 10 equivalents, preferably 1.5 to 5 equivalents, and more preferably 2 to 3 equivalents to the amount of the tetradentate ligand.

The compound of the present invention is preferably produced under inert gas atmosphere. Examples of the inert gas include nitrogen and argon gases, and the like. In addition, the platinum complex is also prepared favorably by using an ultrasonic generator or a microwave generator additionally.

The reaction temperature is properly selected in the range of normally 25 to 300° C., preferably 80 to 250° C., and more preferably 120 to 200° C.

The reaction time may vary according to the reaction conditions such as reaction temperature, solvent, and additives, and is selected in the range of normally 10 minutes to 72 hours, preferably 30 minutes to 48 hours, and more preferably 1 to 12 hours.

The platinum complex of the present invention thus obtained may be posttreated, isolated, and purified as needed. The posttreatment methods include, for example, extraction of reaction product, filtration of precipitate, crystallization by addition of solvent, distillation of solvent, and the like; and these posttreatment methods may be used alone or in combination thereof. Examples of the isolation and purification methods include column chromatography, recrystallization, sublimation, and the like; and they may be used alone or in combination thereof.

The tetradentate ligand of the present invention can be prepared in suitable combination of the synthetic reactions such as carbon/carbon bond-forming reactions by using a palladium catalyst such as Suzuki coupling, Negishi coupling, Sonogashira coupling and Stille coupling; carbon/carbon bond-forming reactions by using a nickel catalyst such as Kumada coupling; carbon/nitrogen bond-forming reactions by using a palladium catalyst; carbon/nitrogen bond-forming reactions by using a copper catalyst such as Ullmann coupling; aromatic ring- and aromatic heterocyclic ring-forming reactions by using a cobalt catalyst; aliphatic and aromatic heterocyclic ring-forming reactions by condensation of a nitrogen-containing compound; halogenation reactions by using, for example, bromine, 1,1,2,2-tetrafluoro-1,2-dibromoethane, N-bromosuccinimide or tetrabutylammonium tribromide; Sandmeyer reactions by using a diazonium salt; lithiation reactions by using an alkyllithium or lithium amide reagent; nucleophilic addition/addition elimination reactions by using an organic lithium reagent or a Grignard reagent; electrophilic aromatic substitution reactions such as Friedel-Crafts reaction; quantitative/catalytic oxidation reactions; quantitative/catalytic reductive reactions; and transfer reactions such as sigmatropic transfer. The tetradentate ligand of the present invention also has a characteristic that it is possible to prepare various kinds of derivatives according to the combination of the reagents and reactions used.

Hereinafter, the light-emitting device of the present invention will be described in detail.

The light-emitting device of the present invention is characterized by that at least one platinum complex of the present invention is contained therein. It is not particularly limited by the system, driving method, and application of the light-emitting device of the present invention are not limited as long as the platinum complex of the present invention is used in the device, and a light-emitting device utilizing the emission from the platinum complex above or utilizing the platinum complex above as a charge-transporting material is preferred. A typical example of such a light-emitting device is organic electroluminescent device (organic EL device).

The light-emitting device of the present invention may be any light-emitting device as long as it is one containing at least one platinum complex of the present invention. When the light-emitting device is prepared by forming a light-emitting layer or multiple organic compound layers including a light-emitting layer between a pair of electrodes, the light-emitting device is characterized by that at least one of the platinum complexes above is contained in at least one layer thereof. The platinum complexes may be contained in combination of two or more thereof as needed.

The method of forming an organic compound layer in the light-emitting device of the present invention is not particularly limited. Examples thereof include methods such as a resistance-heating vapor deposition method, an electron beam method, a sputtering method, a molecular lamination method, a coating method and an inkjet method. Of these, the resistance-heating vapor deposition, coating, and inkjet methods are preferred from viewpoints of properties and productivity of the layer.

The light-emitting device of the present invention is preferably an organic electroluminescent device having a light-emitting layer or multiple organic compound layers including a light-emitting layer between a pair of electrodes, anode and cathode. Examples of the organic compound layers include, in addition to the light-emitting layer, a hole injection layer, a hole transporting layer, an electron injection layer, an electron transporting layer, a protecting layer, and the like; and each of these layers may have other functions as well. Various materials can be used in forming each layer. Hereinafter, each layer will be described in more detail.

The anode supplies holes to the hole injection layer, the hole transporting layer, the light-emitting layers, and the like. And the anode is made of a material such as a metal, an alloy, a metal oxide, an electrically conductive compound, or the mixture thereof. As the material, a material having a work function of 4 eV or more is preferred. Typical examples of the material include electrically conductive metal oxides such as tin oxide, zinc oxide, indium oxide and indium tin oxide (hereinafter, referred to as ITO), metals such as gold, silver, chromium and nickel, mixtures or laminates of the metal above and the electrically conductive metal oxide, inorganic conductive substances such as copper iodide and copper sulfide, organic conductive substances such as polyaniline, polythiophene and polypyrrole, lamination layers of an inorganic/organic conductive substance and ITO, and the like. Of these, electrically conductive metal oxides are preferred, and ITO is particularly preferable from viewpoints, for example, of productivity, high conductivity and transparency.

The thickness of the anode is properly decided according to the material used, and is selected in the range of preferably 10 nm to 5 μm, more preferably 50 nm to 1 μm, and still more preferably 100 nm to 500 nm. The anode normally used is formed as a layer on a material such as soda lime glass, nonalkali glass or transparent resin substrate. When a glass is used, use of a nonalkali glass as the anode substrate is preferable, because of reducing the amount of ions eluted from the glass. Alternatively, soda lime glass, if used, is preferably barrier-coated, for example, with silica. The thickness of the substrate is not particularly limited if it is sufficient for preserving a desirable mechanical strength, and is normally 0.2 mm or more, preferably 0.7 mm or more, when a glass is used. Various methods may be used for preparation of the anode. When ITO is used as an anode material, the ITO anode layer is formed by a method such as an electron beam method, a sputtering method, a resistance-heating vapor deposition method, a chemical reaction method, or a coating method. Reduction of the drive voltage and improvement in the luminous efficiency of the device may be achieved by the cleaning or other processing of the anode. For example, UV-ozone treatment, plasma treatment, and the like are effective in processing ITO anodes. It is preferable that the sheet resistance of the anode is lower.

On the other hand, the cathode supplies electrons to the electron injection layer, the electron transporting layer, the light-emitting layer, and the like; and is selected, considering the adhesiveness to the layer next to the cathode such as an electron injection layer, an electron transporting layer or a light-emitting layer, ionization potential, and stability thereof. As the material for the cathode, a metal, an alloy, a metal halide, a metal oxide, an electrically conductive compound, or the mixture thereof may be used; and typical examples of the materials include alkali metals such as lithium, sodium and potassium and the fluorides thereof, alkali-earth metals such as magnesium and calcium and the fluorides thereof, metals such as gold, silver, lead, aluminum and indium, rare earth metals such as ytterbium, mixed metals such as sodium-potassium alloy, lithium-aluminum alloy and magnesium-silver alloy, and the like. As the material, a material having a work function of 4 eV or more is preferred, and examples of more preferable materials include aluminum, an alloy of lithium and aluminum, an alloy of magnesium and silver, the mixed metal thereof, or the like. The cathode may have a lamination structure containing therein the compound above or the mixture thereof.

The thickness of the cathode may be selected properly according to the material used, and is selected in the range of preferably 10 nm to 5 μm, more preferably 50 nm to 1 μm, and still more preferably 100 nm to 1 μm. The cathode is formed by a method such as an electron beam method, a sputtering method, a resistance-heating vapor deposition method, or a coating method; and a single metal deposition or two or more component simultaneous deposition method may be used in the vapor deposition. Alternatively, an alloy cathode can be formed by simultaneous vapor deposition of multiple metals, or alternatively, by vapor deposition of an alloy previously prepared. It is preferable that the sheet resistance of the cathode is lower.

The material for the light-emitting layer is not particularly limited, if it can form a layer having a function of receive electrons from the anode, the hole injection layer and the hole transporting layer and a function of providing a site for recombination of the holes and the electrons to emit light, when an electric field is applied. Typical examples of thereof include carbazole derivatives, arylamine derivatives, styrylamine derivatives, benzoxazole derivatives, benzothiazole derivatives, benzimidazole derivatives, oxadiazole derivatives, coumarin derivatives, perynone derivatives, naphthalimide derivatives, aldazine derivatives, pyrralizine derivatives, quinacridone derivatives, pyrrolopyridine derivatives, thiadiazopyridine derivatives, oligophenylene derivatives, styrylbenzene derivatives, diphenylbutadiene derivatives, tetraphenylbutadiene derivatives, bisstyrylanthracene derivatives, perylene derivatives, cyclopentadiene derivatives, aromatic dimethylidene compounds, arylborane derivatives, arylsilane derivatives, various typical, transition or rare-earth metal complexes including metal complexes with an 8-quinolinol derivative as the ligand, polymer or oligomer compounds such as poly(N-vinylcarbazole), polythiophene, polyphenylene, and polyphenylene vinylene, the tetradentate ligands of the present invention, the platinum complexes of the present invention, and the like. Each of the polymer or oligomer compounds may have the tetradentate ligand of the present invention or the platinum complex of the present invention as its partial structure independently. The materials for the light-emitting layer are not limited to the typical examples exemplified above.

The light-emitting layer may have a single-layered structure containing one or more of the materials above or a multilayer structure having multiple layers same or different in composition. The thickness of the light-emitting layer is not particularly limited, and is selected in the range of preferably 1 nm to 5 μm, more preferably 5 nm to 1 μm, and still more preferably 10 to 500 nm. The method of forming the light-emitting layer is not particularly limited, and examples thereof include an electron beam method, a sputtering method, a resistance-heating vapor deposition method, a molecular lamination method, a coating method, an inkjet method, and a LB method; and preferred are the resistance-heating vapor deposition method and the coating method.

Typical examples of the coating method include a spin coating method, a casting method, a dip coating method, and the like. A light-emitting layer is formed by dissolving or dispersing the light-emitting layer material above in a solvent and then coating the resulting solution or dispersion by the coating method. At this time, the material may be dissolved or dispersed together with a resin component. Examples of the resin components include polyvinylchloride, polycarbonate, polystyrene, polymethyl methacrylate, polybutyl methacrylate, polyester, polysulfone, polyphenylene oxide, polybutadiene, poly(N-vinylcarbazole), hydrocarbon resins, ketone resins, phenoxy resins, polyamide, ethylcellulose, vinyl acetate resins, ABS resins, alkyd resins, epoxy resins, silicone resins, and the like.

The material for the hole injection layer and hole transporting layer is not particularly limited, if it has a function of receiving holes from the anode, a function of transporting the holes, or a function of blocking the electrons injected from the cathode. Typical examples thereof include, and are not limited to, carbazole derivatives, arylamine derivatives, styrylamine derivatives, phenylenediamine derivatives, amino-substituted chalcone derivatives, hydrazone derivatives, silazane derivatives, oxazole derivatives, imidazole derivatives, pyrazoline derivatives, pyrazolone derivatives, oxadiazole derivatives, triazole derivatives, polyarylalkane derivatives, stilbene derivatives, styrylanthracene derivatives, fluorenone derivatives, aromatic dimethylidene compounds, porphyrin derivatives, phthalocyanine derivatives, arylborane derivatives, arylsilane derivatives, conductive polymer or oligomer compounds such as poly(N-vinylcarbazole), aniline copolymers, polythiophenes, thiophene oligomers, polysilanes, and silane oligomers, the tetradentate ligands of the present invention, the platinum complexes of the present invention, and the like.

The thickness of the hole injection layer or the hole transporting layer is not particularly limited, and is selected in the range of preferably 1 nm to 5 μm, more preferably 5 nm to 1 μm, and still more preferably 10 to 500 nm. The hole injection layer or the hole transporting layer may have a single-layered structure of one or more of the materials described above or a multilayer structure having multiple layers same or different in composition. Examples of the method of forming the hole injection layer or the hole transporting layer include an electron beam method, a sputtering method, a resistance-heating vapor deposition method, a molecular lamination method, a coating method, an inkjet method, and a LB method, and the like; and preferred are the resistance-heating vapor deposition method and the coating method. In the coating method, a hole injection/transporting material may be dissolved or dispersed together with the resin component described above.

The material for the electron injection layer or the electron transporting layer is not particularly limited, if it has a function of receiving electrons from the cathode, a function of transporting the electrons, or a function of blocking the holes injected from the anode. When an electron injection/transporting material is used for blocking the holes injected from the anode, a material having higher ionization potential than that of the light-emitting layer is preferably selected.

Typical examples thereof include oxazole derivatives, oxadiazole derivatives, triazole derivatives, distyrylpyrazine derivatives, bipyridine derivatives, phenanthroline derivatives, carbodiimide derivatives, fluorenone derivatives, anthrone derivatives, diphenylquinone derivatives, thiopyranedioxide derivatives, anthraquinonedimethane derivatives, fluorenylidenemethane derivatives, aromatic tetracarboxylic acid anhydride derivatives, phthalocyanine derivatives, arylborane derivatives, arylsilane derivatives, various typical, transition or rare-earth metal complexes including metal complexes with an 8-quinolinol derivative, a benzoxazole derivative or a benzothiazole derivative as the ligand, polymer or oligomer compounds such as poly(N-vinylcarbazole), polythiophene, polyphenylene, and polyphenylene vinylene, the tetradentate ligands of the present invention, the platinum complexes of the present invention, and the like. Each of the polymer or oligomer compounds may have the tetradentate ligand of the present invention or the platinum complex of the present invention as its partial structure independently. The materials for the electron injection layer or the electron transporting layer are not limited to the materials above.

The thickness of the electron injection layer or the electron transporting layer is not particularly limited, and is selected in the range of preferably 1 nm to 5 μm, more preferably 5 nm to 1 μm, and still more preferably 10 nm to 500 nm. The electron injection layer or the electron transporting layer may have a single-layered structure of one or more of the materials described above or a multilayer structure having multiple layers same or different in composition. Examples of the method of forming the electron injection layer or the electron transporting layer include an electron beam method, a sputtering method, a resistance-heating vapor deposition method, a molecular lamination method, a coating method, an inkjet method, and a LB method, and the like; and preferable are the resistance-heating vapor deposition method and the coating method. In the coating method, solution or dispersion in which an electron injection/transporting material is dissolved or dispersed together with the resin component described above may be used.

The material for the protecting layer is not particularly limited, if it has a function of preventing molecules accelerating deterioration of the device such as water and oxygen from entering into the device. Typical examples thereof include metals such as indium, tin, lead, gold, silver, copper, aluminum, titanium and nickel; metal oxides such as magnesium oxide, silicon dioxide, dialuminum trioxide, germanium oxide, nickel oxide, calcium oxide, barium oxide, diiron trioxide, diytterbium trioxide and titanium oxide; metal fluorides such as lithium fluoride, magnesium fluoride, calcium fluoride, and aluminum fluoride; polymer compounds such as polyethylene, polypropylene, polymethyl methacrylate, polyimide, polyurea, polytetrafluoroethylene, polychloro-trifluoroethylene, and polydichlorodifluoroethylene; copolymer compounds such as a copolymer of chlorotrifluoroethylene and dichlorodifluoroethylene, copolymers obtained by copolymerization of a monomer mixture containing tetrafluoroethylene and at least one comonomer, and fluorine-containing copolymers having a cyclic structure on the main chain of copolymer; water-absorbing substances having a water absorption of 1% or more and moisture-proof substances having a water absorption of 0.1% or less, and the like.

The method of forming the protecting layer is also not particularly limited, and for example, methods such as a vacuum deposition method, a sputtering method, a reactive sputtering method, a MBE (molecular beam epitaxy) method, a cluster ion beam method, an ion plating method, a plasma polymerization (high-frequency excitation ion plating) method, a plasma CVD method, a laser CVD method, a thermal CVD method, a gas source CVD method, and a coating method are applicable.

### EXAMPLES

Hereinafter, the present invention will be described in detail with reference to Reference Examples and Examples, but it should be understood that the present invention is not limited thereby. In the Reference Examples and Examples, the apparatuses used in determining physical properties are as follows:

¹H-NMR spectrum: NMR spectrometer “DRX-500” (Trade name) manufactured by Bruker Japan Co., Ltd. or NMR spectrometer “GEMINI 2000” (Trade name) manufactured by Varian, Inc.

Internal standard substance: tetramethylsilane or residual undeuterated solvent

Mass spectrometry: Mass spectrometer “POLARIS 9” (Trade name) manufactured by Thermo Electron K.K.

Thermal analysis: Thermal analyzer “TG/DTA6200” (Trade name) manufactured by Seiko Instruments Inc.

### Reference Example 1

### Preparation of 1-(3-chlorophenyl)pyrazole

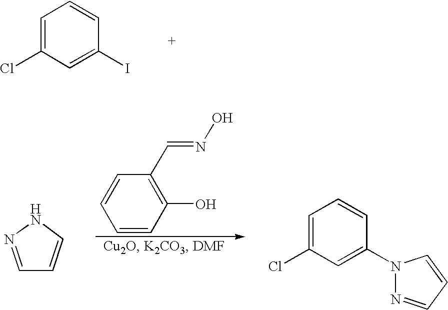

A mixture of pyrazole (5.8 g, 84.8 mmol), potassium carbonate (15.6 g, 113.0 mmol), cuprous oxide (404 mg), salicylaldoxime (1.55 g), 3-chloroiodobenzene (7.0 mL, 56.5 mmol) and N,N-dimethylformamide (20 mL) was stirred under a nitrogen atmosphere at 95° C. for 16 hours. The reaction solution was allowed to cool to room temperature. Then water was added thereto and the mixture was extracted with toluene. The organic phases obtained were combined and concentrated. The residue obtained was purified by silica gel column chromatography, to give 1-(3-chlorophenyl)pyrazole as a pale yellow oily substance (7.6 g). Yield: 75.3%.

¹H-NMR (200 MHz, CDCl₃) δ: 6.48 (t, J=1.8 Hz, 1H), 7.25 (br d, J=8.0 Hz, 1H), 7.37 (t, J=8.0 Hz, 1H), 7.58 (br d, 8.0 Hz, 1H), 7.68-7.80 (m, 2H), and 7.91 (d, J=2.6 Hz, 1H).

### Example 1

### Preparation of N,N-bis[3-(1-pyrazolyl)phenyl]aniline

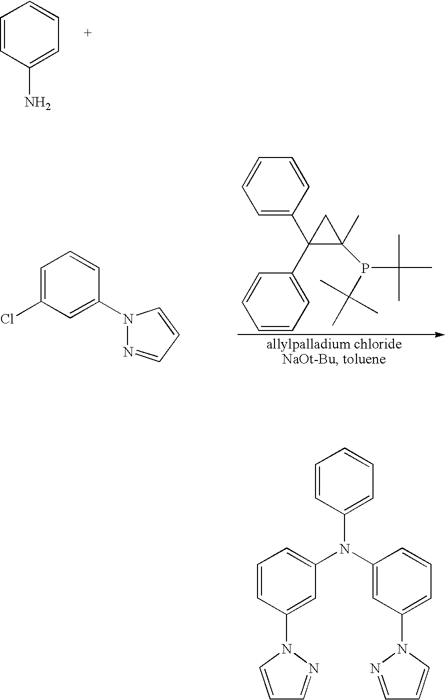

A mixture of aniline (232 μL, 2.55 mmol), 1-(3-chlorophenyl)pyrazole (1.0 g, 5.60 mmol), sodium t-butoxide (613 mg, 6.38 mmol), π-allylpalladium chloride (19 mg), di-t-butyl-(2,2-diphenyl-1-methylcyclopropyl)phosphine (72 mg) and toluene (10 mL) was stirred under a nitrogen atmosphere at 95° C. for 3 hours. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography and recrystallization, to give N,N-bis[3-(1-pyrazolyl)phenyl]aniline as a white powder (883 mg). Yield: 91.7%.

¹H-NMR (200 MHz, CDCl₃) δ: 6.41 (dd, J=2.0, 2.4 Hz, 2H), 6.96-7.22 (m, 6H), 7.24-7.40 (m, 5H), 7.42-7.50 (m, 2H), 7.67 (d, J=2.0 Hz, 2H), and 7.82 (d, J=2.4 Hz, 2H).

### Example 2

### Preparation of Platinum Complex

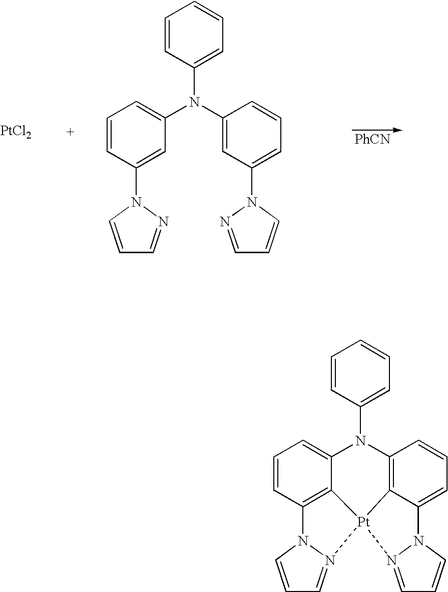

Platinum dichloride (211 mg, 0.795 mmol) and N,N-bis[3-(1-pyrazolyl)phenyl]aniline (300 mg, 0.795 mmol) were allowed to react in benzonitrile (20 mL) in reflux condition under nitrogen atmosphere for 3 hours. The solvent in the reaction solution was distilled off, and the residue obtained was purified by silica gel column chromatography and recrystallization, to give a platinum complex as yellow powder (114 mg). Yield: 25.1%.

¹H-NMR (500 MHz, CD₂Cl₂) δ: 6.04 (dd, J=1.9, 7.4 Hz, 2H), 6.64 (dd, J=2.2, 2.6 Hz, 2H), 6.88-6.94 (m, 4H), 7.30 (dd, J=1.2, 8.4 Hz, 2H), 7.52 (t, J=7.4 Hz, 1H), 7.65 (dd, J=7.4, 9.0 Hz, 2H), 7.89 (dd, J=0.3, 2.1 Hz, 2H), and 8.10 (dd, J=0.3, 2.7 Hz, 2H).

Sublimation temperature: 262.5° C.

Thermal decomposition point: 383.94° C.

### Reference Example 2

### Preparation of 2-(3-chlorophenyl)pyridine

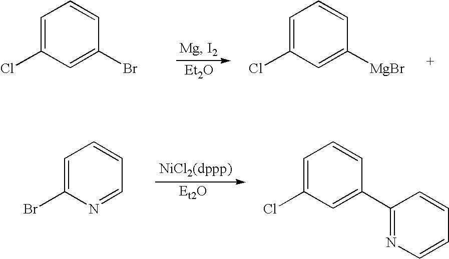

Trace amount of iodine powder was added into a mixture of magnesium (3.46 g) and diethylether (5 mL) under nitrogen atmosphere, and the mixture was stirred until the solution became colorless. Then, a solution of 3-bromochlorobenzene (25.0 g, 130.6 mmol) in diethylether (100 mL) was added dropwise at a speed at which the reaction mixture refluxes gently over a period of 1 hour. The mixture was then stirred additionally for 1 hour under reflux to give a diethylether solution of 3-chlorophenylmagnesium bromide.

Under nitrogen atmosphere, the diethylether solution of 3-chlorophenylmagnesium bromide (130.6 mmol) previously prepared was added dropwise to a mixture of 2-bromopyridine (11.3 mL, 118.7 mmol), [1,3-bis(diphenylphosphino)propane]nickel dichloride (643 mg) and diethylether (100 mL), at a speed at which the reaction mixture refluxes gently for 30 minute. The mixture was then stirred additionally for 1 hour under reflux and allowed to cool to room temperature. The reaction solution was poured into aqueous ammonium chloride-saturated solution, and the mixture was extracted with methylene chloride. The organic phases were combined and concentrated, and the residue obtained was purified by silica gel column chromatography and distillation, to give 2-(3-chlorophenyl)pyridine as a colorless oily substance (19.2 g). Yield: 85.3%.

¹H-NMR (200 MHz, CDCl₃) δ: 7.27 (ddd, J=1.6, 4.6, 7.0 Hz, 1H), 7.66-7.94 (m, 3H), 8.01 (br s, 1H), and 8.70 (d, J=4.6 Hz, 1H).

### Reference Example 3

### Preparation of N-[3-(1-pyrazolyl)phenyl]aniline

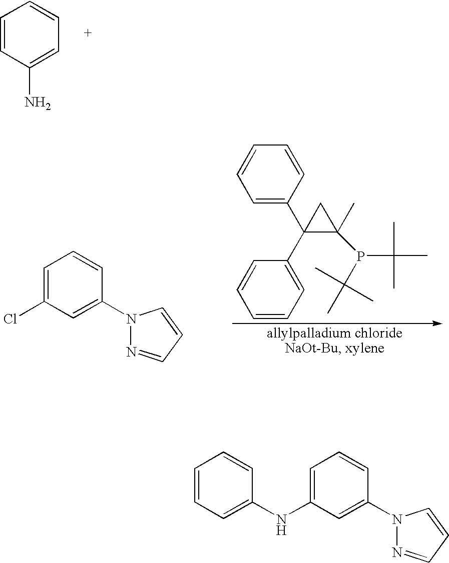

A mixture of aniline (1.1 mL, 11.8 mmol), 1-(3-chlorophenyl)pyrazole (2.0 g, 11.2 mmol), sodium t-butoxide (1.3 g, 13.4 mmol), π-allylpalladium chloride (41 mg), di-t-butyl-(2,2-diphenyl-1-methylcyclopropyl)phosphine (158 mg) and xylene (40 mL) was stirred under a nitrogen atmosphere at 95° C. for 3 hours. The reaction solution was allowed to cool to room temperature and aqueous ammonium chloride-saturated solution was added thereto. Then the mixture was extracted with toluene and the organic phases obtained were combined and concentrated. The residue obtained was purified by silica gel column chromatography, to give N-[3-(1-pyrazolyl)phenyl]aniline as an yellow viscous oily substance (2.2 g). Yield: 83.5%.

¹H-NMR (200 MHz, CDCl₃) δ: 5.85 (br s, 1H), 6.44 (dd, J=1.8, 2.6 Hz, 1H), 6.92-7.05 (m, 2H), 7.08-7.22 (m, 3H), 7.24-7.38 (m, 3H), 7.43 (t, J=2.2 Hz, 1H), 7.70 (d, J=1.8 Hz, 1H), and 7.88 (dd, J=0.8, 2.6 Hz, 1H).

### Example 3

### Preparation of N-[3-(1-pyrazolyl)phenyl]-N-[3-(2-pyridyl)phenyl]-aniline

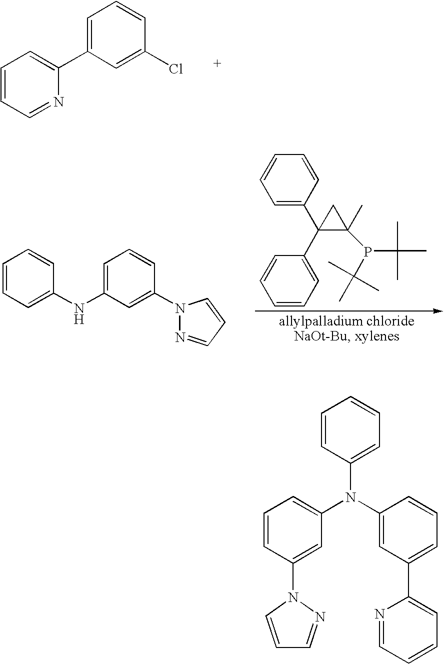

A mixture of 2-(3-chlorophenyl)pyridine (846 mg, 4.46 mmol), N-[3-(1-pyrazolyl)phenyl]aniline (1.0 g, 4.25 mmol), sodium t-butoxide (490 mg, 5.10 mmol), π-allylpalladium chloride (16 mg), di-t-butyl-(2,2-diphenyl-1-methylcyclopropyl)phosphine (60 mg) and xylene (20 mL) was stirred under a nitrogen atmosphere at 100° C. for 4 hours. The reaction solution was allowed to cool to room temperature and aqueous ammonium chloride-saturated solution was added thereto. The mixture was extracted with toluene and the organic phases obtained were combined and concentrated. The residue obtained was purified by silica gel column chromatography, to give N-[3-(1-pyrazolyl)phenyl]-N-[3-(2-pyridyl)phenyl]aniline as an yellow amorphous substance (1.7 g). Yield: 99.9%.

¹H-NMR (500 MHz, CDCl₃) δ: 6.40 (t, J=2.0 Hz, 1H), 6.96-7.10 (m, 2H), 7.12-7.48 (m, 10H), 7.56-7.84 (m, 6H), and 8.63 (br d, 5.0 Hz, 1H).

### Example 4

### Preparation of Platinum Complex

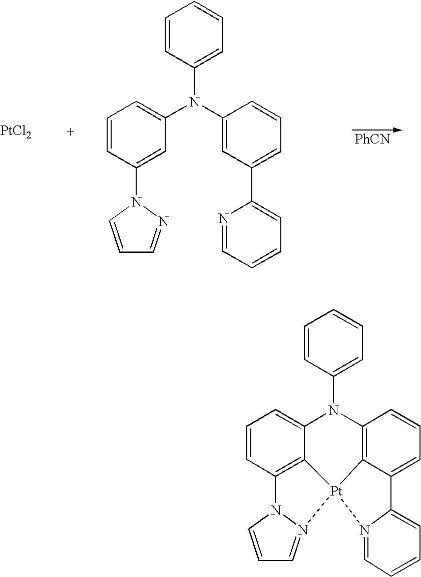

Platinum dichloride (326 mg, 1.23 mmol) and N-[3-(1-pyrazolyl)phenyl]-N-[3-(2-pyridyl)phenyl]aniline (500 mg, 1.29 mmol) was stirred in benzonitrile (50 mL) in reflux condition under nitrogen atmosphere for 4 hours. The solvent in the reaction solution was distilled off and the residue obtained was purified by silica gel column chromatography and recrystallization, to give a platinum complex as orange powder (420 mg). Yield: 58.7%.

¹H-NMR (500 MHz, CD₂Cl₂) δ: 6.09 (dd, J=1.2, 8.1 Hz, 1H), 6.23 (dd, J=0.9, 8.4 Hz, 1H), 6.70 (dd, J=2.2, 2.7 Hz, 1H), 6.90-7.02 (m, 3H), 7.29-7.35 (m, 3H), 7.37 (ddd, J=1.8, 5.5, 7.2 Hz, 1H), 7.50-7.56 (m, 1H), 7.62-7.72 (m, 2H), 7.88-7.96 (m, 2H), 7.97 (d, J=2.0 Hz, 1H), 8.17 (dd, J=0.3, 2.7 Hz, 1H), and 8.97 (ddd, J=1.0, 1.4, 5.5 Hz, 1H).

Sublimation temperature: 288.9° C.

Thermal decomposition point: 415.0° C.

### Reference Example 4

### Preparation of 2-(3-chlorophenyl)thiazole

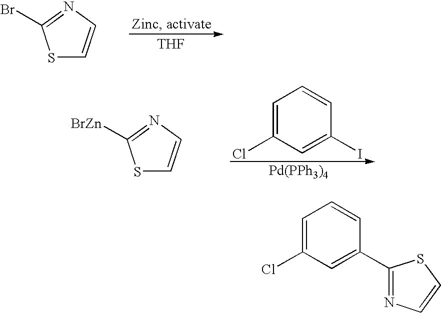

1,2-Dibromobutane (717 μL) was added to a suspension of zinc powder (10.9 g) in tetrahydrofuran (10 mL). The mixture was heated under reflux for 5 minutes and then chlorotrimethylsilane (1.1 mL) was added thereto. Then, a tetrahydrofuran (50 mL) solution of 2-bromothiazole (5.0 mL, 55.5 mmol) was added dropwise and the mixture was stirred at 50° C. for 1 hour to give a tetrahydrofuran solution of 2-thiazolylzinc bromide. 3-Chloroiodobenzene (6.2 mL, 50.5 mmol) and tetrakis(triphenylphosphine)palladium (584 mg) were added sequentially to the solution obtained and the mixture was stirred at 60° C. for 12 hours. The reaction solution was poured into aqueous sodium bicarbonate-saturated solution (500 mL) containing ethylenediaminetetraacetic acid (16.2 g) and the mixture was extracted with toluene and the organic phases obtained were combined and concentrated. The residue obtained was purified by silica gel column chromatography and recrystallization, to give 2-(3-chlorophenyl)thiazole as a white powder (9.1 g). Yield: 92.1%.

¹H-NMR (200 MHz, CDCl₃) δ: 7.32-7.44 (m, 3H), 7.80-7.87 (m, 1H), 7.89 (d, J=3.2 Hz, 1H), and 7.99 (br s, 1H).

### Example 5

### Preparation of N-[3-(1-pyrazolyl)phenyl]-N-[3-(2-thiazolyl)-phenyl]aniline

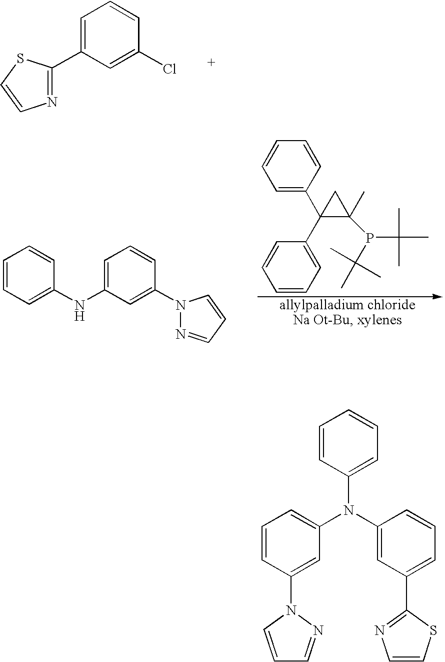

A mixture of 2-(3-chlorophenyl)thiazole (873 mg, 4.46 mmol), N-[3-(1-pyrazolyl)phenyl]aniline (1.0 g, 4.25 mmol), sodium t-butoxide (490 mg, 5.10 mmol), π-allylpalladium chloride (16 mg), di-t-butyl-(2,2-diphenyl-1-methylcyclopropyl)phosphine (60 mg) and xylene (20 mL) was stirred under a nitrogen atmosphere at 100° C. for 4 hours. The reaction solution was allowed to cool to room temperature and aqueous ammonium chloride-saturated solution was added thereto. Then the mixture was extracted with toluene and the organic phases obtained were combined and concentrated. The residue obtained was purified by silica gel column chromatography to give N-[3-(1-pyrazolyl)phenyl)-N-[3-(2-thiazolyl)phenyl]aniline as an yellow amorphous material (1.7 g). The yield was quantitative.

¹H-NMR (200 MHz, CDCl₃) δ: 6.40 (t, J=2.2 Hz, 1H), 6.96-7.21 (m, 6H), 7.27-7.40 (m, 5H), 7.44 (br s, 1H), 7.61 (dt, J=7.6, 1.4 Hz, 1H), 7.66 (d, J=1.8 Hz, 1H), 7.76 (t, J=2.0 Hz, 1H), and 7.78-7.84 (m, 2H).

### Example 6

### Preparation of Platinum Complex

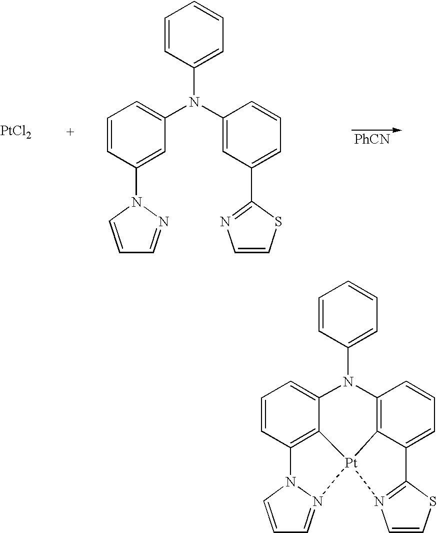

Platinum dichloride (321 mg, 1.21 mmol) and N-[3-(1-pyrazolyl)phenyl]-N-[3-(2-thiazolyl)phenyl]aniline (500 mg, 1.27 mmol) were stirred in benzonitrile (50 mL) in reflux condition under a nitrogen atmosphere for 3 hours. The solvent in the reaction solution was distilled off, and the residue obtained was purified by silica gel column chromatography and recrystallization to give a platinum complex as orange powder (171 mg). Yield: 24.0%.

¹H-NMR (500 MHz, CD₂Cl₂) δ: 6.05 (dd, J=2.3, 6.9 Hz, 1H), 6.17 (dd, J=0.9, 8.4 Hz, 1H), 6.66 (dd, J=2.2, 2.6 Hz, 1H), 6.89-6.96 (m, 3H), 7.21 (dd, J=0.8, 7.2 Hz, 1H), 7.28-7.34 (m, 2H), 7.46 (d, J=3.4 Hz, 1H), 7.50-7.56 (m, 1H), 7.63-7.70 (m, 2H), 7.92 (d, J=2.2 Hz, 1H), 7.98 (d, J=3.4 Hz, 1H), and 8.11 (dd, J=0.4, 2.8 Hz, 1H).

Sublimation temperature: 285.3° C.

Thermal decomposition point: 381.52° C.

### Reference Example 5

### Preparation of 3,3′-dibromobenzophenone

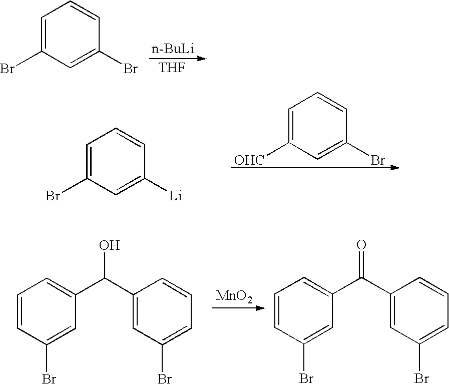

Under a nitrogen atomosphere, a tetrahydrofuran solution (10 mL) of 1,3-dibromobenzene (1.9 mL, 16.1 mmol) was cooled to −70° C. and then n-butyllithium (10.0 mL, 1.60 N, 16.1 mmol) was added dropwise thereto over a period of 15 minutes. After the mixture was stirred at −70° C. additionally for 20 minutes, a tetrahydrofuran (10 mL) solution of 3-bromobenzaldehyde (1.7 mL, 14.6 mmol) was added dropwise over a period of 15 minutes, and the mixture after the dropwise addition was allowed to warm to room temperature. The reaction solution was poured into aqueous ammonium chloride-saturated solution, and the mixture was extracted with toluene. The organic phases obtained were combined and concentrated to give 1,1-bis(3-bromophenyl)methanol as a colorless oily substance. The substance was used in the next reaction without further purification.

Manganese dioxide (14.2 g, 146.0 mmol) was added to a methylene chloride (70 mL) solution of 1,1-bis(3-bromophenyl)methanol (14.6 mmol) and the mixture was stirred at room temperature in air for 1 hour. The reaction solution was filtered. The filtrate was concentrated and the residue obtained was purified by silica gel column chromatography and recrystallization to give 3,3′-dibromobenzophenone as white powder (3.5 g). Yield: 70.5%.

¹H-NMR (200 MHz, CDCl₃) δ: 7.38 (t, J=8.0 Hz, 2H), 7.65-7.79 (m, 4H), and 7.93 (dd, J=1.6, 2.0 Hz, 2H).

### Example 7

### Preparation of carbonylbis[3-(1-pyrazolyl)benzene]

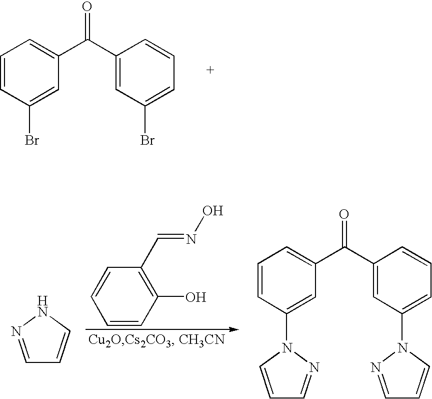

A mixture of 3,3′-dibromobenzophenone (3.0 g, 8.8 mmol), pyrazole (1.5 g, 22.1 mmol), cesium carbonate (8.6 g, 26.5 mmol), cuprous oxide (126 mg), salicylaldoxime (484 mg) and acetonitrile (20 mL) was stirred in reflux condition under nitrogen atmosphere for 24 hours. The reaction solution obtained was allowed to cool to room temperature. Water and toluene were added thereto and the extraction was carried out. The organic phases obtained were combined and concentrated. The residue obtained was purified by silica gel column chromatography and recrystallization to give carbonyl bis[3-(1-pyrazolyl)benzene] as white powder (1.8 g). Yield: 64.9%.

¹H-NMR (200 MHz, CDCl₃) δ: 6.51 (dt, J=0.6, 1.8 Hz, 2H), 7.60 (t, J=7.7 Hz, 2H), 7.68-7.78 (m, 4H), 7.98-8.08 (m, 4H), and 8.12-8.18 (m, 2H).

### Example 8

### Preparation of Platinum Complex

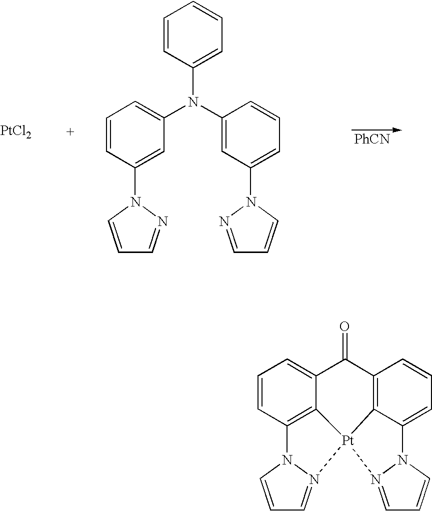

Platinum dichloride (423 mg, 1.59 mmol) and carbonyl bis[3-(1-pyrazolyl)benzene] (500 mg, 1.59 mmol) were allowed to react in benzonitrile (40 mL) in reflux condition under nitrogen atmosphere for 8 hours. After the reaction solution was allowed to cool, methylene chloride was added thereto. The crystal precipitated was filtered and purified by sublimation to give a platinum complex as yellow powder (300 mg). Yield: 37.2%.

Mass Spectrum (EI): m/z=507 (M⁺)

Sublimation temperature: 319.9° C.

Thermal decomposition point: 457.8° C.

### Example 9

### Preparation of 9,9-bis[3-(1-pyrazolyl)phenyl]-9H-fluorene

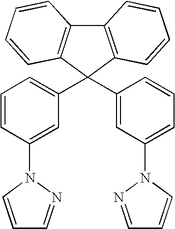

¹H-NMR (200 MHz, CDCl₃) δ: 6.38 (t, J=2.1 Hz, 2H), 7.16 (d, J=7.8 Hz, 2H), 7.28-7.62 (m, 12H), 7.65 (d, J=1.8 Hz, 2H), 7.73 (d, J=2.6 Hz, 2H), and 7.80 (d, J=6.8 Hz, 2H).

### Example 10

### Preparation of N,N-bis[6-(1-pyrrolyl)pyridine-2-yl]aniline

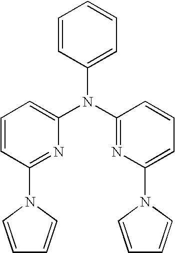

¹H-NMR (200 MHz, CDCl₃) δ: 6.25 (t, J=2.3 Hz, 4H), 6.89 (d, J=4.8 Hz, 2H), 6.93 (d, J=4.4 Hz, 2H), 7.24-7.38 (m, 7H), 7.40-7.50 (m, 2H), and 7.61 (t, J=8.0 Hz, 2H).

As apparent from the results in Examples 2, 4, 6, and 8, each of the platinum complexes of the present invention has high thermal stability.

### Example 11

### Preparation of Organic EL Device

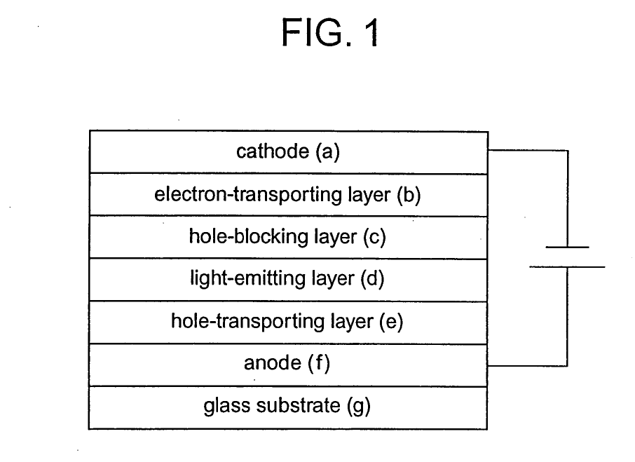

An organic EL device having the layer structure shown in FIG. 1 was prepared by forming an anode (f), a hole transporting layer (e), a light-emitting layer (d) comprising a host material and a dope material, a hole blocking layer (c), an electron transporting layer (b) and a cathode (a) on a glass plate (g), sequentially in the order from the glass plate (g) side. In the organic EL device, each of the anode (f) and the cathode (a) is connected to a lead wire, and voltage can be applied between the anode (f) and cathode (a). Specific materials and preparative methods for each layer will be described below.

First, the anode (f) is an ITO film and bonded onto the glass plate (g). The hole transporting layer (e) was formed by vacuum deposition of 4,4′-bis[N-(1-naphthyl)-N-phenylamino]biphenyl (α-NPD) represented by the following Formula on the anode (f) to a thickness of 40 nm.

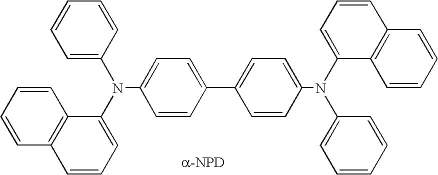

The light-emitting layer (d) was formed on the hole transporting layer (e) to a thickness of 35 nm by vacuum co-deposition of 4,4′-bis(9H-carbazole-9-yl)biphenyl (CBP) and the platinum complex obtained in Example 2 (platinum complex-doping amount: 6 wt %) represented by the following Formulae.

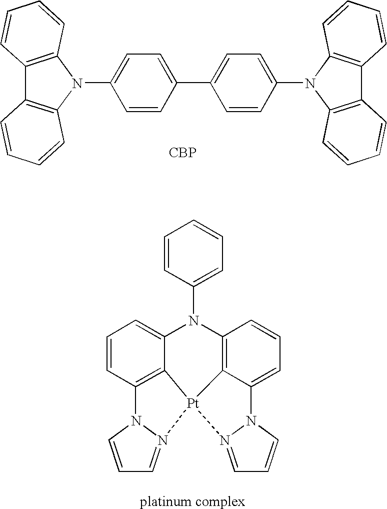

The hole blocking layer (c) was formed on the light-emitting layer (d) to a thickness of 10 nm by vacuum deposition of 2,9-dimethyl-4,7-diphenyl-1,10-phenanthroline (BCP) represented by the following Formula.

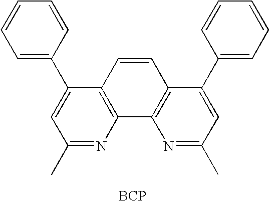

The electron transporting layer (b) was formed on the hole blocking layer (c) to a thickness of 35 nm by vapor deposition of tris(8-quinolinolato-O,N] aluminum (Alq₃) represented by the following Formula.

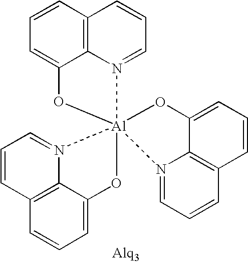

The cathode (a) was formed as a laminate film by vacuum deposition of lithium fluoride to a thickness of 0.5 nm and metal aluminum to a thickness of 100 nm, sequentially from the electron transporting layer (b) side.

Application of a plus voltage to the anode (f) and a minus voltage to the cathode (a) of the organic EL device thus prepared resulted in stabilized light emission even at a very low voltage. At a brightness of 100 cd/m², the external quantum efficiency of the device was 6.0 (%); the power efficiency was 6.5 (lm/W); the brightness-current efficiency was 15.8 (cd/A); and the maximum external quantum efficiency was 7.3%, indicating that the device has high efficiency. In addition, the device gave a blue green emission derived from the platinum complex obtained in Example 2, which was used in the light-emitting layer (d), and showed the emission peak of 491 (nm) and the CIE chromaticity point (x, y) of (0.201, 0.462) at a brightness of 100 cd/m².

### Example 12

An organic EL device having an device configuration similar to that in Example 11 was prepared in a similar manner to Example 11, except that 4,4′-bis(9H-carbazole-9-yl)-2,2′-dimethylbiphenyl (CDBP) represented by the following Formula was used in the light-emitting layer (d).

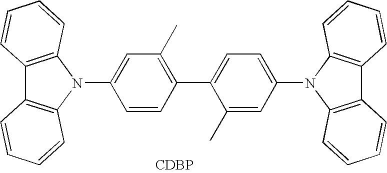

Physical properties of the device were determined in a similar manner to Example 11. At a brightness of 100 cd/m², the external quantum efficiency of the device was 9.1 (%); the power efficiency was 7.6 (lm/W); the brightness-current efficiency was 19.5 (cd/A); and the maximum external quantum efficiency was 11.4%, indicating that the device had an extremely high efficiency. In addition, the device gave a pale blue to blue green emission derived from the platinum complex obtained in Example 2, which was used in the light-emitting layer (d), and showed the emission peak of 486 (nm) and the CIE chromaticity point (x, y) of (0.196, 0.430) at a brightness of 100 cd/m².

### Example 13

An organic EL device having an device configuration similar to that in Example 11 was prepared in a similar manner to Example 11, except that CBP and the below platinum complex obtained in Example 4 (platinum complex-doping amount: 6 wt %) were used in the light-emitting layer (d) and bis(2-methyl-8-quinolinolato-O,N)-4-phenylphenolato-aluminum (BAlq) shown below was used as the hole blocking layer (c).

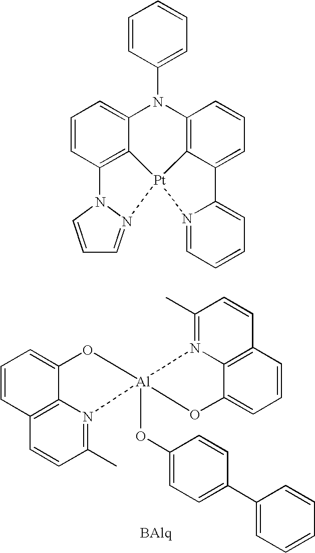

Physical properties of the device were determined in a similar manner to Example 11. At a brightness 100 cd/m², the external quantum efficiency of the device was 9.4 (%); the power efficiency was 8.6 (lm/W); the brightness-current efficiency was 22.4 (cd/A); and the maximum external quantum efficiency was 10.4%, indicating that the device had an extremely high efficiency. In addition, the device gave an orange emission derived from the platinum complex obtained in Example 4, which was used in the light-emitting layer (d), and showed the emission peak of 582 (nm) and the CIE chromaticity point (x, y) of (0.549, 0.450) at a brightness of 100 cd/m².

### Example 14

An organic EL device having a device configuration similar to that in Example 11 was prepared in a similar manner to Example 11, except that CBP and the platinum complex obtained in Example 6 (platinum complex-doping amount: 6 wt %) showed below were used in the light-emitting layer (d).

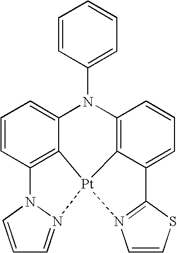

Physical properties of the device were determined in a similar manner to Example 11. At a brightness 100 cd/m², the external quantum efficiency of the device was 8.0 (%); the power efficiency was 5.2 (lm/W); the brightness-current efficiency was 12.8 (cd/A); and the maximum external quantum efficiency was 8.3%, indicating that the device had an extremely high efficiency. In addition, the device gave a vermeil emission derived from the platinum complex obtained in Example 4, which was used in the light-emitting layer (d), and showed the emission peak of 604 (nm) and the CIE chromaticity point (x, y) of (0.601, 0.391) at a brightness of 100 cd/m².

Results obtained in Examples 11 to 14 are summarized in the following Tables 1 and 2.

TABLE 1
Characteristics of the EL device prepared (at 100 cd/m²)

| | | | | Maximum |
|---|---|---|---|---|
| | External | | | external |
| | quantum | Power | Luminance-Current | quantum |
| Ex. | efficiency | efficiency | efficiency | efficiency |
| No. | (%) | (lm/W) | (cd/A) | (%) |
| 11 | 6.0 | 6.5 | 15.8 | 7.3 |
| 12 | 9.1 | 7.6 | 19.5 | 11.4 |
| 13 | 9.4 | 8.6 | 22.4 | 10.4 |
| 14 | 8.0 | 5.2 | 12.8 | 8.3 |

TABLE 2
Emission color

| Ex. | Emission | |
|---|---|---|
| No. | peak (nm) | CIE chromaticity |
| 11 | 491 | (0.201, 0.462) |
| 12 | 486 | (0.196, 0.430) |
| 13 | 582 | (0.549, 0.450) |
| 14 | 604 | (0.601, 0.391) |

As apparent from the results in Examples 11 to 14, each of the organic EL devices containing the platinum complex of the present invention has light-emitting characteristics and a luminous efficiency better than the limit in the external quantum efficiency of fluorescence emitting materials, and gave an emission derived from the platinum complex used, which was different in color in the range from shorter wavelength (blue) to longer wavelength (red).

The results in Examples described above also indicate that the platinum complexes of the present invention are superior in thermal stability, light-emitting characteristics and luminous efficiency, and are favorably applicable to various light-emitting devices including organic EL devices. In addition, the light-emitting devices containing the platinum complex of the present invention are also superior in light-emitting characteristics and luminous efficiency. Further, the light-emitting devices are favorably applicable to various display devices as giving various emission colors from shorter wavelength (blue) to longer wavelength (red) derived from the platinum complex used.

What is claimed is:

1. A platinum complex represented by General Formula (1): 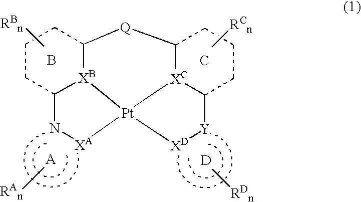
wherein two of the rings A, B, C, and D each independently represent an aromatic ring or an aromatic heterocyclic ring, while the other two rings each independently represent a nitrogen-containing heterocyclic ring; each of the rings B and C is always a six-membered ring independently of the kind of its ring; R^{A}, R^{B}, R^{C}, and R^{D} respectively represent substituents on the rings A, B, C, and D; the rings A and B, the rings B and C, and the rings C and D each may be bound each other via the substituent R^{A}, R^{B}, R^{C} or R^{D} to form a fused ring independently; X^{A}, X^{B}, X^{C}, and X^{D} each independently represent a carbon atom that can be bound with the platinum atom by a covalent bond or a nitrogen atom that can be bound with the platinum atom by a covalent bond when the corresponding ring is an aromatic ring or an aromatic heterocyclic ring, and a nitrogen atom that can be bound with the platinum atom by a coordinate bond when the corresponding ring is a nitrogen-containing heterocyclic ring; Q represents a bivalent atom or atomic group bridging the rings B and C; the ring B and Q, and the ring C and Q each independently may be bound each other via a substituent R^{B} or R^{C} to form a fused ring; Y represents a carbon atom or a nitrogen atom; n is an integer of 0 to 3; and when n is 2 or more, the groups R^{A}, the groups R^{B}, the groups R^{C}, and the groups R^{D} each independently may be bound each other to form a fused ring.

2. The platinum complex according to claim 1, wherein aromatic or aromatic heterocyclic rings in the compound represented by General Formula (1) each independently represent a ring selected from the group consisting of benzene, furan, thiophene, selenophene, tellurophene, pyrrole, pyridine, pyridazine, pyrimidine, pyrazine, 1,2,3-triazine, 1,2,4-triazine, 1,2,3,4-tetrazine, oxazole, isoxazole, thiazole, isothiazole, pyrazole, imidazole, 1,2,3-oxadiazole, 1,2,5-oxadiazole, 1,2,3-thiadiazole, 1,2,5-thiadiazole, triazole and tetrazole rings, which may have a substituent or substituents and may form a fused ring with any ring selected from the aforementioned group.

3. The platinum complex according to claim 1, wherein nitrogen-containing heterocyclic rings in the compound represented by General Formula (1) each independently represent a ring selected from the group consisting of pyridine, pyridazine, pyrimidine, pyrazine, triazine, tetrazine, 2H-pyrrole, 3H-pyrrole, oxazole, isoxazole, thiazole, isothiazole, pyrazole, imidazole, oxadiazole, thiadiazole, triazole, oxatriazole, thiatriazole, tetrazole, 2H-3,4-dihydropyrrole, oxazoline, isooxazoline, thiazoline, isothiazoline, pyrazoline and imidazoline rings, which may have a substituent or substituents, and may form a fused ring with any ring selected from the aromatic rings or the aromatic heterocyclic rings described in claim 2.

4. The platinum complex according to claim 1, wherein group Q in the compounds represented by General Formula (1) represents a bivalent atom or atomic group selected from an oxy group, a thio group, a seleno group, a telluro group, a sulfinyl group, a sulfonyl group, an imino group which may have a substituent, a phosphinidene group which may have a substituent, a phosphinylidene group which may have a substituent, a methylene group which may have a substituent or substituents, an alkenylidene group which may have a substituent or substituents, a carbonimidoyl group which may have a substituent, a carbonyl group, a thiocarbonyl group, a silylene group which may have a substituent or substituents and a borylene group which may have a substituent, a bivalent atomic group in which two to five of the atoms or atomic groups may be bound in series or condensed and when plural substituents exist on the atoms and atomic groups the substituents may be bound each other to form a ring.

5. The platinum complex according to claim 1, wherein groups R^{A}, R^{B}, R^{C}, and R^{D} in the compound represented by General Formula (1) each independently represent a group or an atom selected from the group consisting of a hydrocarbyl group, an aliphatic heterocyclic group, an aromatic heterocyclic group, a hydroxyl group, an alkoxy group, an aryloxy group, an aralkyloxy group, a heteroaryloxy group, an acyloxy group, an alkoxycarbonyloxy group, an acyl group, a carboxyl group, an alkoxycarbonyl group, an aryloxycarbonyl group, an aralkyloxycarbonyl group, a heteroaryloxycarbonyl group, a carbamoyl group, a hydroxamic acid group, a mercapto group, an alkylthio group, an arylthio group, an aralkylthio group, a heteroarylthio group, an acylthio group, an alkoxycarbonylthio group, a sulfinyl group, a sulfino group, a sulfenamoyl group, a sulfonyl group, a sulfo group, a sulfamoyl group, an amino group, a hydrazino group, an ureido group, a nitro group, a phosphino group, a phosphinyl group, a phosphinico group, a phosphono group, a silyl group, a boryl group, a cyano group, and a halogen atom.

6. A light-emitting device containing one or more of the compound represented by General Formula (1) described in claim 1.

7. The light-emitting device according to claim 6, wherein the light-emitting device is one having a light-emitting layer or plural organic compound layers including a light-emitting layer between a pair of electrodes and contains one or more of compounds represented by General Formula (1) in one or more of the layers.

8. The light-emitting device according to claim 7, wherein the light-emitting device is an organic electroluminescent device (organic EL device).

9. The light-emitting device according to claim 8, wherein the compound represented by General Formula (1) and contained in one or more of the layers is able to act as a doping material (guest material) in the light-emitting material of the organic electroluminescent device.

10. A compound represented by General Formula (2): 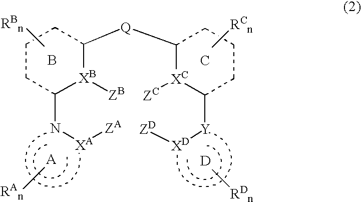
wherein two of the rings A, B, C, and D each independently represent an aromatic ring or an aromatic heterocyclic ring, while the other two rings each independently represent a nitrogen-containing heterocyclic ring; each of the rings B and C is always a six-membered ring independently of the kind of its ring; R^{A}, R^{B}, R^{C}, and R^{D} respectively represent substituents on the rings A, B, C, and D; the rings A and B, the rings B and C, and the rings C and D each may be bound each other via the substituent R^{A}, R^{B}, R^{C}, or R^{D} to form a fused ring independently; X^{A}, X^{B}, X^{C}, and X^{D} each independently represent a carbon atom or a nitrogen atom when the corresponding ring is an aromatic ring or an aromatic heterocyclic ring, and a nitrogen atom when the corresponding ring is a nitrogen-containing heterocyclic ring; Z^{A}, Z^{B}, Z^{C}, and Z^{D} each represent a hydrogen atom when the corresponding X is a carbon atom, and a hydrogen atom or a lone electron pair when the corresponding X is a nitrogen atom; Q represents a bivalent atom or atomic group bridging the rings B and C; the ring B and Q, and the ring C and Q each independently may be bound each other via a substituent R^{B} or R^{C} to form a fused ring; Y represents a carbon atom or a nitrogen atom; n is an integer of 0 to 3; and when n is 2 or more, the groups R^{A}, the groups R^{B}, the groups R^{C}, and the groups R^{D} each may be independently bound each other to form a fused ring.

11. The compound according to claim 10, wherein aromatic or aromatic heterocyclic rings in the compound represented by General Formula (2) each independently represent a ring selected from the group consisting of benzene, furan, thiophene, selenophene, tellurophene, pyrrole, pyridine, pyridazine, pyrimidine, pyrazine, 1,2,3-triazine, 1,2,4-triazine, 1,2,3,4-tetrazine, oxazole, isoxazole, thiazole, isothiazole, pyrazole, imidazole, 1,2,3-oxadiazole, 1,2,5-oxadiazole, 1,2,3-thiadiazole, 1,2,5-thiadiazole, triazole and tetrazole rings which may have a substituent or substituents and may form a fused ring by any ring selected from the aforementioned group.

12. The compound according to claim 11, wherein nitrogen-containing heterocyclic rings in the compound represented by General Formula (2) each independently represent a ring selected from the group consisting of pyridine, pyridazine, pyrimidine, pyrazine, triazine, tetrazine, 2H-pyrrole, 3H-pyrrole, oxazole, isoxazole, thiazole, isothiazole, pyrazole, imidazole, oxadiazole, thiadiazole, triazole, oxatriazole, thiatriazole, tetrazole, 2H-3,4-dihydropyrrole, oxazoline, isooxazoline, thiazoline, isothiazoline, pyrazoline and imidazoline rings, which may have a substituent or substituents, and may form a fused ring with any ring selected from the aromatic and aromatic heterocyclic rings described in claim 11.

13. The compound according to claim 11, wherein group Q in the compounds represented by General Formula (2) represents a bivalent atom or atomic group selected from an oxy group, a thio group, a seleno group, a telluro group, a sulfinyl group, a sulfonyl group, imino group which may have a substituent, a phosphinidene group which may have a substituent, a phosphinylidene group which may have a substituent, a methylene group which may have a substituent or substituents, an alkenylidene group which may have a substituent or substituents, a carbonimidoyl group which may have a substituent, a carbonyl group, a thiocarbonyl group, a silylene group which may have a substituent or substituents and a borylene group which may have a substituent, a bivalent atomic group in which two to five of the atoms or atomic groups may be bound in series or condensed and when plural substituents exist on the atoms and atomic groups the substituents may be bound each other to form a ring.

14. The compound according to claim 11, wherein groups R^{A}, R^{B}, R^{C}, and R^{D} in the compound represented by General Formula (2) each independently represent a group or an atom selected from the group consisting of a hydrocarbyl group, an aliphatic heterocyclic group, an aromatic heterocyclic group, a hydroxyl group, an alkoxy group, an aryloxy group, an aralkyloxy group, a heteroaryloxy group, an acyloxy group, an alkoxycarbonyloxy group, an acyl group, a carboxyl group, an alkoxycarbonyl group, an aryloxycarbonyl group, an aralkyloxycarbonyl group, a heteroaryloxycarbonyl group, a carbamoyl group, a hydroxamic acid group, a mercapto group, an alkylthio group, an arylthio group, an aralkylthio group, a heteroarylthio group, an acylthio group, an alkoxycarbonylthio group, a sulfinyl group, a sulfino group, a sulfenamoyl group, a sulfonyl group, a sulfo group, a sulfamoyl group, an amino group, a hydrazino group, an ureido group, a nitro group, a phosphino group, a phosphinyl group, a phosphinico group, a phosphono group, a silyl group, a boryl group, a cyano group, and a halogen atom.
